(19) United States
(12) Patent Application Publication
(10) Pub. No.: US 2019/0167619 A1
(43) Pub. Date: Jun. 6, 2019

(54) PHARMACEUTICAL FORMULATIONS AND THEIR USE

(71) Applicant: SIGNUM BIOSCIENCES, INC., Princeton, NJ (US)

(72) Inventors: Michael VORONKOV, Pennington, NJ (US); Gareth WINCKLE, Biot (FR)

(73) Assignee: SIGNUM BIOSCIENCES, INC., Princeton, NJ (US)

(21) Appl. No.: 16/323,970

(22) PCT Filed: Aug. 8, 2017
(86) PCT No.: PCT/US17/45945

§ 371 Date: Feb. 7, 2019

Related U.S. Application Data
(60) Provisional application No. 62/372,207, filed on Aug. 8, 2016.

(51) Int. Cl. A61K 31/197; A61K 47/10; A61K 47/02; A61K 47/14

(52) U.S. Cl. CPC A61K 31/197; A61K 47/10; A61K 9/0014; A61K 47/14; A61K 47/02

(57) ABSTRACT

A pharmaceutical composition comprising (a) at least one protective agent selected from the group consisting of butylated hydroxyanisole, butylated hydroxytoluene, sodium metabisulfite, tert-butylhydroquinone, methylparaben, propylparaben, benzyl alcohol, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol, cholesterol, and polyoxyethylene(4)lauryl ether; and (b) a therapeutically effective amount of an IPC Active Agent or a pharmaceutically acceptable salt or ester thereof.

### CROSS-REFERENCE TO RELATED APPLICATIONS

This application claims the benefit of U.S. Provisional Application No. 62/372,207, filed Aug. 8, 2016, the disclosure of which is incorporated herein by reference in its entirety.

### FIELD

The present disclosure relates to pharmaceutical formulations and their use in the treatment of skin conditions in a subject.

### BACKGROUND

Difficulties associated with the treatment of conditions related to bacterial colonization of mammalian epithelium are well-appreciated amongst dermatologists. This is particularly true in the case of skin and wound antisepsis, where the most effective treatment of epithelial conditions caused or aggravated by bacterial colonization, often includes the use of a topical anti-bacterial agent.

Rosacea is a skin condition characterized by inflammatory eruption of the nose and adjoining flush areas of the face. Rosacea is characterized by erythema, papules, pustules, telangiectasia and, frequently, by hypertrophy of the sebaceous glands. Rosacea brings about a flushing of the nose and cheeks and, in some cases, the forehead and chin. In severe forms, lesions appear which are deep or purplish red and which include a chronic dilation of the superficial capillaries, this constituting the above-referenced telangiectasia. Also, in severe form, inflammatory acneiform pustules are present. In such serious conditions, the eye or eyelids may become affected.

Acne vulgaris is a skin condition that occurs when hair follicles become clogged with dead skin cells and oil from the skin. The propionibacterium acnes (P. acnes) bacteria may invade the clogged follicles and grow in the mixture of oil and cells in the hair follicle. Acne is characterized by areas of inflammation, pustules, blackheads, whiteheads, pimples, and greasy skin, deeper lumps such as cysts or nodules and may result in scarring or disfiguring.

Atopic dermatitis, also known as atopic eczema, is a type of inflammation of the skin that results in itchy, red, swollen, and cracked skin. The causes of atopic dermatitis are believed to involve genetics, immune system dysfunction, environmental exposures, and difficulties with the permeability of the skin.

IPC Active Agents (defined below) have been disclosed that are useful in treating, for example, conditions related to bacterial colonization of mammalian epithelium, in U.S. Published Application Nos. 2010/0184768 and 2011/0118265, each of which being hereby incorporated by reference.

For example, U.S. Pat. No. 8,461,204, the contents of which are hereby incorporated by reference in its entirety, discloses the preparation and potential uses of an IPC Active Agent, 4-((1-carboxy-2-(((2E,6E)-3,7,11-trimethyldodeca-2,6,10-trien-1-yl)thio)ethyl)amino)-4-oxobutanoic acid, and pharmaceutically acceptable salts thereof. Formulations of 4-((1-carboxy-2-(((2E,6E)-3,7,11-trimethyldodeca-2,6,10-trien-1-yl)thio)ethyl)amino)-4-oxobutanoic acid, and pharmaceutically acceptable salts thereof, however, may exhibit instability concerns when such formulations are stored.

As such, there is a need to develop improved formulations of 4-((1-carboxy-2-(((2E,6E)-3,7,11-trimethyldodeca-2,6,10-trien-1-yl)thio)ethyl)amino)-4-oxobutanoic acid, and pharmaceutically acceptable salts thereof, that exhibit improved properties to permit their longer-term storage and use.

### SUMMARY

One embodiment of the present invention provides a pharmaceutical composition comprising (a) at least one protective agent selected from the group consisting of butylated hydroxyanisole, butylated hydroxytoluene, sodium metabisulfite, tert-butylhydroquinone, methylparaben, propylparaben, benzyl alcohol, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol, cholesterol, and polyoxyethylene(4)lauryl ether; and (b) a therapeutically effective amount of an IPC Active Agent or a pharmaceutically acceptable salt or ester thereof.

In one embodiment, the protective agent is selected from the group consisting of butylated hydroxyanisole, butylated hydroxytoluene, sodium metabisulfite, tert-butylhydroquinone, methylparaben, propylparaben, and poly(acrylic acid).

In one embodiment, the protective agent includes butylated hydroxyanisole. In one embodiment, the butylated hydroxyanisole is present in an amount from about 0.001% to about 2%, based on the total weight of the composition, or from about 0.005% to about 1%, based on the total weight of the composition.

In one embodiment, the protective agent includes sodium metabisulfite. In one embodiment, the sodium metabisulfite is present in an amount from about 0.01% to about 5%, based on the total weight of the composition, or from about 0.05% to about 1%, based on the total weight of the composition.

In one embodiment, the protective agent includes tert-butylhydroquinone. In one embodiment, the tert-butylhydroquinone is present in an amount from about 0.001% to about 2%, based on the total weight of the composition, or from about from about 0.005% to about 1%, based on the total weight of the composition.

In certain embodiments, the IPC Active Agent is depicted by Formula I:

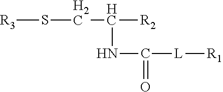

wherein:

L is a bivalent, branched or unbranched, saturated or unsaturated, C₂-C₆ hydrocarbon chain wherein one or more methylene units of L is independently replaced by —O—, —S—, —NH—, —C(O)—, —C═CH₂—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;

R₁ is hydrogen, —OH or —OR, wherein each R is independently hydrogen or an optionally substituted group selected from C₁₋₆ aliphatic or C₁₋₆ heteroaliphatic;

R₂ is —C(O)X, wherein X is independently R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic; and

R₃ is a substituted or unsubstituted, branched or unbranched, saturated or unsaturated, C₁₀-C₂₅ aliphatic,

or a pharmaceutically acceptable salt or ester thereof.

In certain embodiments, the IPC Active Agent includes 4-((1-carboxy-2-(((2E,6E)-3,7,11-trimethyldodeca-2,6,10-trien-1-yl)thio)ethyl)amino)-4-oxobutanoic acid or a pharmaceutically acceptable salt or ester thereof. In one embodiment the IPC Active Agent includes the disodium salt of 4-((1-carboxy-2-(((2E,6E)-3,7,11-trimethyldodeca-2,6,10-trien-1-yl)thio)ethyl)amino)-4-oxobutanoic acid. In one embodiment, the IPC Active Agent includes 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof. In one embodiment, the IPC Active Agent includes the disodium salt of 4-(((R)-1-carboxy-2-(((2E,6E)-3,7,11-trimethyldodeca-2,6,10-trien-1-yl)thio)ethyl)amino)-4-oxobutanoic acid.

In certain embodiments, the IPC Active Agent includes 4-((1-carboxy-2-(((2E,6E)-3,7,11-trimethyldodeca-2,6,10-trien-1-yl)thio)ethyl)amino)-4-oxobutanoic acid or a pharmaceutically acceptable salt or ester thereof. In one embodiment the IPC Active Agent includes the disodium salt of 4-((1-carboxy-2-(((2E,6E)-3,7,11-trimethyldodeca-2,6,10-trien-1-yl)thio)ethyl)amino)-4-oxobutanoic acid.

In one embodiment, the IPC Active Agent includes 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof. In one embodiment, the IPC Active Agent includes the disodium salt of 4-(((R)-1-carboxy-2-(((2E,6E)-3,7,11-trimethyldodeca-2,6,10-trien-1-yl)thio)ethyl)amino)-4-oxobutanoic acid.

In one embodiment, the IPC Active Agent includes 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof. In one embodiment, the IPC Active Agent includes the disodium salt of 4-(((S)-1-carboxy-2-(((2E,6E)-3,7,11-trimethyldodeca-2,6,10-trien-1-yl)thio)ethyl)amino)-4-oxobutanoic acid.

In one embodiment, the IPC Active Agent is:

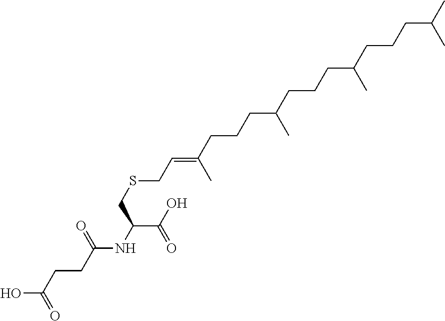 [SMILES: C/C(=C\CSC[C@H](NC(=O)CCC(=O)O)C(=O)O)CCCC(C)CCCC(C)CCCC(C)C]

or a pharmaceutically acceptable salt or ester thereof.

In one embodiment, the IPC Active Agent is:

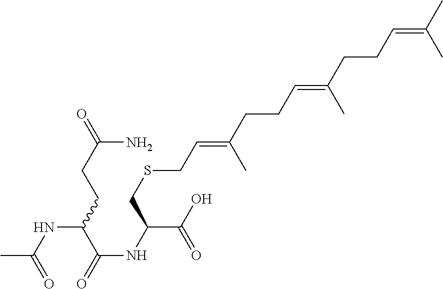 [SMILES: CC(=O)NC(CCC(N)=O)C(=O)N[C@@H](CSC/C=C(\C)CC/C=C(\C)CCC=C(C)C)C(=O)O]

or a pharmaceutically acceptable salt or ester thereof.

### DETAILED DESCRIPTION

As used herein, the term “butylated hydroxyanisole” or “BHA” refers to a protective agent that includes one or more of 2-tert-butyl-4-hydroxyanisole and 3-tert-butyl-4-hydroxyanisole. In certain embodiments, butylated hydroxyanisole can include a mixture of both 2-tert-butyl-4-hydroxyanisole and 3-tert-butyl-4-hydroxyanisole.

As used herein, the term “butylated hydroxytoluene” or “BHT” refers to a protective agent that includes the compound:

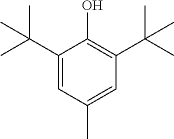 [SMILES: Cc1cc(C(C)(C)C)c(O)c(C(C)(C)C)c1]

2,6-di-tert-butyl-4-methylphenol.

As used herein, the term “tert-butylhydroquinone” or “TBHQ” refers to a protective agent that includes a hydroquinone substituted with a tert-butyl group, including the compound:

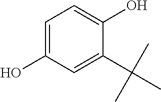

2-(tert-butyl)benzene-1,4-diol.

As used herein, the term “sodium metabisulfite” refers to a protective agent that includes the compound:

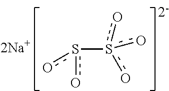

As used herein, the term “diethylene glycol monoethyl ether” refers to a protective agent that includes 2-(2-Ethoxyethoxy)ethanol, preferably a composition that contains purified 2-(2-Ethoxyethoxy)ethanol (e.g., at least 99% pure 2-(2-Ethoxyethoxy)ethanol). Examples of diethylene glycol monoethyl ether include, but are not limited to, compositions known as carbitol, 3,6-dioxa-1-octanol, diethylene glycol ethyl ether, diglycol monoethyl ether, dioxitol, ethanol, 2,2-oxybis-, monoethyl ether, ethyl carbitol, ethyl diethylene glycol, ethyl digol; and compositions commercially sold under the trademarks Dowanol 17, Dowanol DE, Ektasolve DE, Solvolsol, Transcutol, Transcutol P, and Transcutol HP.

As used herein, the term “polysorbate 80” refers to a protective agent that includes polyoxyethylene (20) sorbitan monooleate. Polysorbate 80 is also know as, for example, E433, and is commercially sold under the trademarks Alkest TW 80, Scattics, Canarcel, Poegasorb 80 and Tween 80.

As used herein, the term “poly(acrylic acid)” or “PAA” or “carbomer” refers to a synthetic high molecular weight polymers of acrylic acid, such as crosslinked polyacrylate polymers and acrylate/C₁₀-C₃₀ alkyl acrylate crosspolymers. Examples, of poly(acrylic acid) include but are not limited to, compositions commercially sold under the trademark Carbopol 940, Carbopol 980, Carbopol 981 and Pemulen TR-1.

The term “hydroxyethyl cellulose” includes pharmaceutical grades of hydroxyethylcellulose. In certain embodiments, the hydroxyethylcellulose is a freeflowing granular powder that can be of high molecular weight, or ultra-high molecular weight, and/or a fine grind particle size. Examples of hydroxyethyl cellulose include commercially available hydroxyethylcellulose sold under the trademark Natrosol 250 (e.g. Natrosol 250 HHX PHARM).

As used herein, the term “4-((1-carboxy-2-(((2E,6E)-3,7,11-trimethyldodeca-2,6,10-trien-1-yl)thio)ethyl)amino)-4-oxobutanoic acid” means a compound having the chemical structure:

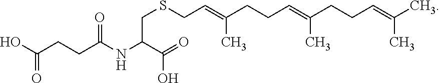 [SMILES: CC(C)=CCC/C(C)=C/CC/C(C)=C/CSCC(NC(=O)CCC(=O)O)C(=O)O]

As used herein, the term “4-(((R)-1-carboxy-2-(((2E,6E)-3,7,11-trimethyldodeca-2,6,10-trien-1-yl)thio)ethyl)amino)-4-oxobutanoic acid” means a compound having the chemical structure”

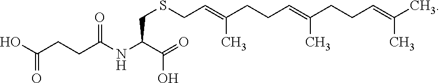 [SMILES: CC(C)=CCC/C(C)=C/CC/C(C)=C/CSC[C@H](NC(=O)CCC(=O)O)C(=O)O]

As used herein, the term “4-(((S)-1-carboxy-2-(((2E,6E)-3,7,11-trimethyldodeca-2,6,10-trien-1-yl)thio)ethyl)amino)-4-oxobutanoic acid” means a compound having the chemical structure:

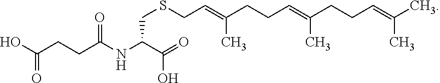 [SMILES: CC(C)=CCC/C(C)=C/CC/C(C)=C/CSC[C@@H](NC(=O)CCC(=O)O)C(=O)O]

The preparation of compounds 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, and 4-(((S)-1-carboxy-2-(((2E,6E)-3,7,11-trimethyldodeca-2,6,10-trien-1-yl)thio)ethyl)amino)-4-oxobutanoic acid, and pharmaceutically acceptable salts thereof, are disclosed in U.S. Pat. Nos. 8,372,884 and 8,461,204, the contents of which are hereby incorporated by reference in their entirety.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, the term “about” means either within plus or minus 10% of the provided value, or rounded to the nearest significant figure, in all cases inclusive of the provided value. Where ranges are provided, they are inclusive of the boundary values.

As used herein, the terms “administration” and “administering” mean the delivery of a bioactive composition or formulation by an administration route including, but not limited to, intravenous, intra-arterial, intramuscular, intraperitoneal, subcutaneous, intramuscular, topically, or combinations thereof.

As used herein, the term “antioxidant” means an agent, such as a chemical element or compound, that reduces or prevents the chemical oxidation of a second chemical element or compound.

As used herein, the terms “combination” and “in combination with” mean the administration of one or more compounds disclosed herein, or a pharmaceutically acceptable salt or ester thereof together with an at least one additional pharmaceutical or medicinal agent (e.g., an anti-cancer agent), either sequentially or simultaneously. It includes dosing simultaneously, or within minutes or hours of each other, or on the same day, or on alternating days, or dosing the compound disclosed herein on a daily basis, or multiple days per week, or weekly basis, for example, while administering another compound such as a chemotherapeutic agent on the same day or alternating days or weeks or on a periodic basis during a time simultaneous therewith or concurrent therewith, or at least a part of the time during which the compound disclosed herein is dosed. For example, one or more compounds disclosed herein, or a pharmaceutically acceptable salt or ester thereof, or a pharmaceutically acceptable salt or ester thereof, could be dosed every day or several days a week while the chemotherapeutic agent is dosed on alternating days or alternating weeks or other periods of time, such as every 1, 2, 3, 4, 5, 6, 7, 8, 9, 10, 11. 12, 13, 14 or more days.

As used herein, the term “degradation” means a change in the chemical structure of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) resulting from one or more chemical reactions.

As used herein, the term “lithium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one of the carboxylic acid moieties in the compound is deprotonated to afford a carboxylate anion that is complexed with a lithium counterion.

As used herein, the term “dilithium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which both of the carboxylic acid moieties in the compound are deprotonated to afford carboxylate anions that are complexed with lithium counterions.

As used herein, the term “sodium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one of the carboxylic acid moieties in the compound is deprotonated to afford a carboxylate anion that is complexed with a sodium counterion.

As used herein, the term “disodium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which both of the carboxylic acid moieties in the compound are deprotonated to afford carboxylate anions that are complexed with sodium counterions.

As used herein, the term “potassium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one of the carboxylic acid moieties in the compound is deprotonated to afford a carboxylate anion that is complexed with a potassium counterion.

As used herein, the term “dipotassium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which both of the carboxylic acid moieties in the compound are deprotonated to afford carboxylate anions that are complexed with potassium counterions.

As used herein, the term “calcium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one or more of the carboxylic acid moieties in the compound is deprotonated to afford one or more carboxylate anions, as the case may be, that are complexed with a calcium counterion.

As used herein, the term “magnesium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one or more of the carboxylic acid moieties in the compound is deprotonated to afford one or more carboxylate anions, as the case may be, that are complexed with a magnesium counterion.

As used herein, the term “strontium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one or more of the carboxylic acid moieties in the compound is deprotonated to afford one or more carboxylate anions, as the case may be, that are complexed with a strontium counterion.

As used herein, the term “barium salt” means a salt form of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a mixture thereof, as the case may be) in which one or more of the carboxylic acid moieties in the compound is deprotonated to afford one or more carboxylate anions, as the case may be, that are complexed with a barium counterion.

As used herein, the term “oxidation” means the chemical oxidation of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt, or a pharmaceutically acceptable ester, or a mixture thereof). For example, 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt, or a pharmaceutically acceptable ester, or a mixture thereof, may undergo oxidation in which the sulfur atom in the compounds, or pharmaceutically acceptable salts thereof, or a mixture thereof, is converted to higher oxidation state, such as the oxidation state of sulfur found in a sulfoxide or a sulfone, by means of one more chemical reactions.

As used herein, the term “pharmaceutically acceptable salt” means those salts that retain the biological effectiveness and properties of the parent compound.

As used herein, the term “pharmaceutically acceptable ester” means those esters that retain the biological effectiveness and properties of the parent compound.

As used herein, the term “protective agent” means a first chemical compound or element that reduces or prevents the degradation of a second chemical compound, such as degradation of the second chemical compound by oxidation or other chemical reaction, or otherwise assists with the chemical and/or physical stability of the second chemical compound (e.g., an IPC Active Agent) over a period of time. It is understood that components can have multiple functions. Accordingly, a particularly component can be a protective agent, while also being disclosed in this application to have another function. For example, a component that is identified as an excipient can also be a protective agent.

### IPC Active Agents

As used herein, the term “IPC” refers to compounds containing cysteine and one or more isoprenoid chains, such as phytyl, farnesyl or geranylgeranyl groups. As used herein, the term “IPC Active Agents” are IPC compounds that are pharmaceutically active and can be used to treat a disease or condition. In certain embodiments, IPC Active Agents are structurally related to N-acetyl-5-farnesyl-L-cysteine (AFC), and includes AFC itself, along with any pharmaceutically acceptable salts or esters thereof.

In one embodiment, the IPC Active Agent is represented by Formula I:

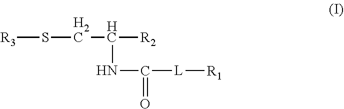

wherein:

L is a bivalent, branched or unbranched, saturated or unsaturated, C₂-C₆ hydrocarbon chain wherein one or more methylene units of L is independently replaced by —O—, —S—, —NH—, —C(O)—, —C═CH₂—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;

R₁ is hydrogen, —OH or —OR, wherein each R is independently hydrogen or an optionally substituted group selected from C₁₋₆ aliphatic or C₁₋₆ heteroaliphatic;

R₂ is —C(O)X, wherein X is independently R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic; and

R₃ is a substituted or unsubstituted, branched or unbranched, saturated or unsaturated, C₁₀-C₂₅ aliphatic,

or a pharmaceutically acceptable salt or ester thereof.

In one embodiment, the IPC Active Agent is represented by Formula Ia:

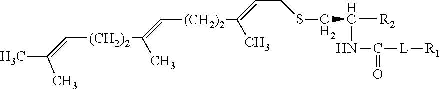

wherein:

L is a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;

R₁ is hydrogen, —OH or —OR, wherein each R is independently hydrogen or an optionally substituted group selected from C₁-C₆ aliphatic or C₁-C₆ heteroaliphatic; and

R₂ is —C(O)X, wherein X is independently R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁-C₆ aliphatic or C₁-C₆ heteroaliphatic,

or a pharmaceutically acceptable salt or ester thereof.

In one embodiment, the IPC Active Agent includes any one of the compounds specifically depicted and/or encompassed by genus formulas disclosed in U.S. Published Patent Application No. 2010/0184768, which is hereby incorporated by reference.

In one embodiment, the IPC Active Agent is:

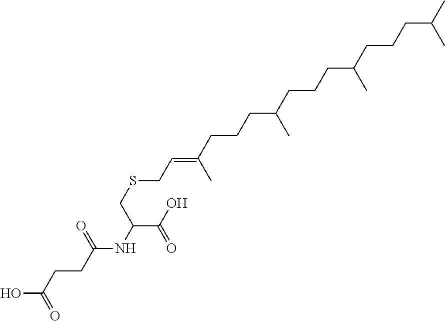 [SMILES: C/C(=C\CSCC(NC(=O)CCC(=O)O)C(=O)O)CCCC(C)CCCC(C)CCCC(C)C]

or a pharmaceutically acceptable salt or ester thereof.

In one embodiment, the IPC Active Agent is:

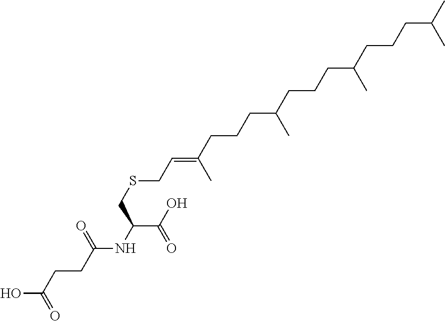 [SMILES: C/C(=C\CSC[C@H](NC(=O)CCC(=O)O)C(=O)O)CCCC(C)CCCC(C)CCCC(C)C]

or a pharmaceutically acceptable salt or ester thereof.

In one embodiment, the IPC Active Agent is:

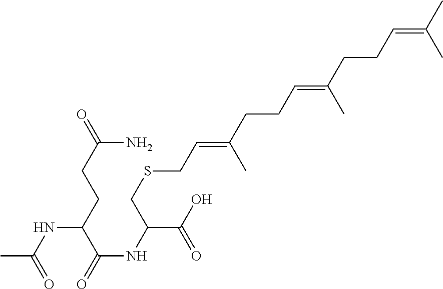 [SMILES: CC(=O)NC(CCC(N)=O)C(=O)NC(CSC/C=C(\C)CC/C=C(\C)CCC=C(C)C)C(=O)O]

or a pharmaceutically acceptable salt or ester thereof.

In one embodiment, the IPC Active Agent is:

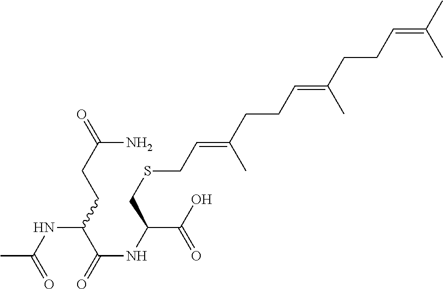 [SMILES: CC(=O)NC(CCC(N)=O)C(=O)N[C@@H](CSC/C=C(\C)CC/C=C(\C)CCC=C(C)C)C(=O)O]

or a pharmaceutically acceptable salt or ester thereof.

In one embodiment, the IPC Active Agent is selected from the group consisting of Compounds A-N-98, as disclosed in Table 1 of U.S. Published Application No. 2010/0184768, which is hereby incorporated by reference.

In one embodiment, the IPC Active Agent includes any one of the active agents specifically depicted and/or encompassed by genus formulas disclosed in U.S. Published Patent Application No. 2011/0118265, which is hereby incorporated by reference.

In one embodiment, the IPC Active Agent is represented by the formula:

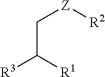

wherein:
R¹ is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN; substituted or unsubstituted heteroaryl; or one of the following moieties:

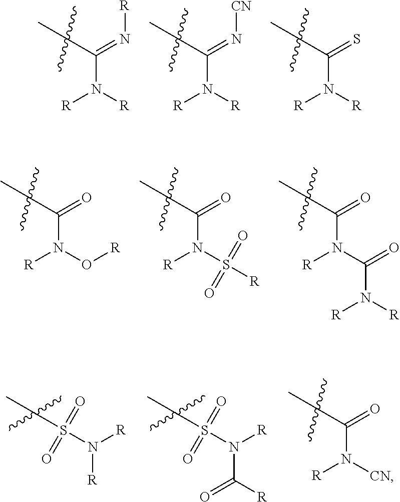

wherein each R is independently hydrogen or an optionally substituted group selected from C₁-C₆ aliphatic, C₁-C₆ heteroaliphatic, aryl, heteroaryl, or a cyclic radical;
R² is a substituted or unsubstituted, branched or unbranched C₁₀-C₂₅ aliphatic moiety;
R³ is —NH₂, a peptide, or —N(R⁴)(R⁵);
R⁴ is hydrogen or an optionally substituted group selected from C₁-C₆ aliphatic, C₁-C₆ heteroaliphatic, a cyclic radical, aryl or heteroaryl;
R₅ is heteroaryl; —C(═N—R⁶)(R⁷), wherein R⁶ is selected from hydrogen, aliphatic, and —N(R)₂, and R⁷ is selected from hydrogen, aliphatic, aryl, cyano, and —SO₂R; or C(O)LR⁸, wherein L is a covalent bond or a bivalent, branched or unbranched, saturated or unsaturated, C₂-C₆ hydrocarbon chain wherein one or more methylene units of L is independently replaced by —O—, —S—, —NH—, —C(O)—, —C(═CH₂)—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur; and R₈ is —R, —OR, —N(R)₂, a cyclic radical, aryl, heteroaryl, wherein each R is independently hydrogen or an optionally substituted group selected from C₁-C₆ aliphatic, C₁-C₆ heteroaliphatic, aryl, heteroaryl, or a cyclic radical; or a substituted or unsubstituted peptidic moiety; and
Z is —S—, —O—, —NH—, —Se—, —S(═O)—, —S(═N)—, —SO₂—, —Se(═O)—, or —SeO₂—.

In one embodiment, the IPC Active Agent is represented by the formula:

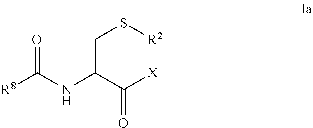

wherein R² is a substituted or unsubstituted, branched or unbranched C₁₀-C₂₅ aliphatic moiety;
X is —OH, halogen, methyl, —SH, —NH₂, or —N(R)₂, wherein R is hydrogen or C₁-C₃ alkyl; and
R⁸ is C₁-C₃ alkyl.

In one embodiment, the IPC Active Agent is represented by the formula:

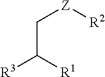

wherein
R¹ is —CO₂H, —CO₂R, —CONH₂, —NO₂, —PO₃H, —CN, or —SO₃H, where R is as defined herein;
R² is farnesyl, phytyl, geranylgeranyl, substituted farnesyl, substituted phytyl, or substituted geranylgeranyl; and
R³ is —NH₂ or a peptide.

In one embodiment, the IPC Active Agent is represented by the formula:

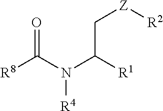

wherein R² is is farnesyl, phytyl, geranylgeranyl, substituted farnesyl, substituted phytyl, or substituted geranylgeranyl and R⁸ is C₁-C₃ alkyl;
R¹ is substituted or unsubstituted heteroaryl, or one of the following moieties:

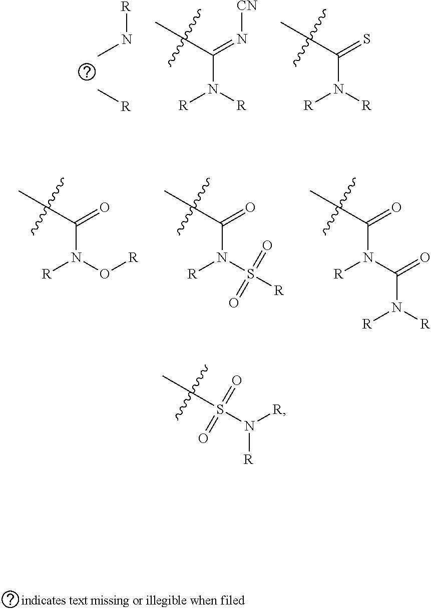

wherein R is independently hydrogen or an optionally substituted group selected from C₁-C₆ aliphatic, C₁-C₆ heteroaliphatic, aryl, heteroaryl, or a cyclic radical; and

### Z is —S—, —O—, —Se—, —SO—, —SO₂—, or —NH—.

In one embodiment, the IPC Active Agent is represented by the formula:

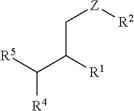

wherein R² and R⁴ are as described anywhere herein;
substituted or unsubstituted heteroaryl, or one of the following moieties

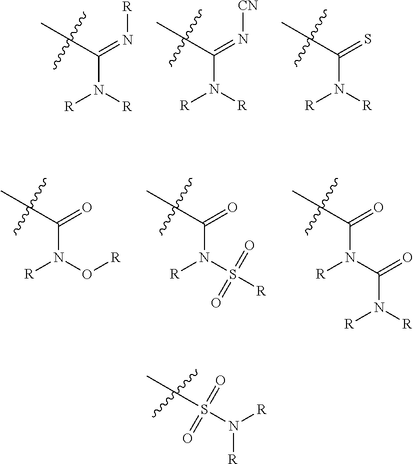

wherein R is as described anywhere herein;
R⁵ is heteroaryl or —C(═NR⁶)(R⁷), where R⁶ and R⁷ are as described anywhere herein; and

### Z is —S—, —O—, —Se—, —SO—, —SO₂—, or —NH—.

In one embodiment, the IPC Active Agent is represented by the formula:

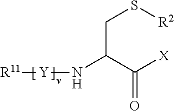

wherein
Y is a natural or unnatural amino acid;
v is an integer between 1 and 100, inclusive; and
R¹¹ is hydrogen, a protecting group, or an optionally substituted group selected from C₁-C₆ aliphatic, C₁-C₆ heteroaliphatic, aryl or heteroaryl.

In one embodiment, the IPC Active Agent is represented by the formula:

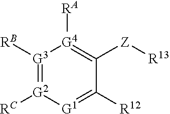

wherein each of G¹, G², G³, and G⁴ is N or CR^{D};

### Z is S, O, Se, SO, SO₂, or NH;

R¹² is —C(O)X, wherein X is independently a protecting group, a halogen, R, —OR, —SR, —N(R)₂, a substituted or unsubstituted hydrazine, a substituted or unsubstituted 6-10 membered aryl ring, a substituted or unsubstituted 5-6 membered heteroaryl ring having 1-4 heteroatoms independently selected from nitrogen, oxygen, or sulfur; —NO₂; —PO₃H; —SO₃H; —CN;
substituted or unsubstituted heteroaryl; or one of the following moieties:

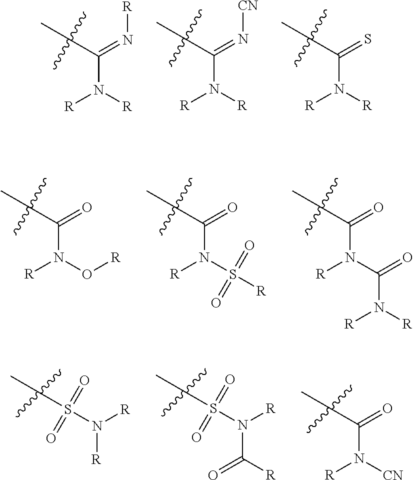

wherein each R is independently hydrogen or an optionally substituted group selected from C₁-C₆ aliphatic, C₁-C₆ heteroaliphatic, aryl, heteroaryl, or a cyclic radical;
R¹³ is an optionally substituted aliphatic group;
R^{A}, R^{B}, R^{C}, and R^{D} are independently H, —NO₂, —OR¹⁴, halogen, alkylN(R¹⁴)₂, —N(R¹⁴)₂, —C(═O)R¹⁴, —C(═O)OR¹⁴, —S(R¹⁴), azido, —S—C═N, alkyl, aryl, alkenyl, alkynyl, or a cyclic radical, wherein R^{A}, R^{B}, R^{C}, and R^{D} are further optionally substituted;
R¹⁴ is H, alkyl, aryl, alkenyl, alkynyl, or a cyclic radical, wherein R¹⁴ is further optionally substituted.

In some embodiments, at least one of G¹, G², G³, and G⁴ is N; in some embodiments, at least two of G¹, G², G³, and G⁴ are N; in some embodiments, at least three of G¹, G², G³, and G⁴ are N; in some embodiments, at least four of G¹, G², G³, and G⁴ are N. In some embodiments, G¹ is N. In some embodiments, G¹ is N and at least one of G², G³, and G⁴ is N.

In one embodiment, the IPC Active Agent is selected from the group consisting of Compounds A-M, as disclosed in Table 1 of U.S. Published Application No. 2011/0118265, which is hereby incorporated by reference. In one embodiment, the present invention provides pharmaceutical compositions comprising a therapeutically effective amount of an IPC Active Agent, as defined herein, and at least one protective agent.

Solely for purposes of convenience, IPC Active Agents are described below largely in relation to 4-((1-carboxy-2-(((2E,6E)-3,7,11-trimethyldodeca-2,6,10-trien-1-yl)thio)ethyl)amino)-4-oxobutanoic acid, or pharmaceutically acceptable salts or esters thereof, yet it is understood that every such reference to 4-((1-carboxy-2-(((2E,6E)-3,7,11-trimethyldodeca-2,6,10-trien-1-yl)thio)ethyl)amino)-4-oxobutanoic acid anywhere in this application, including the Examples, is taken also to be a reference to any one of the IPC Active Agents disclosed herein, including IPC Active Agents specifically depicted and/or encompassed by genus formulas disclosed in U.S. Published Patent Application No. 2010/0184768, U.S. Published Application No. 2011/0118265, and/or U.S. Published Application No. 2012/0328540, each of which hereby being incorporated by reference in their entirety as if it were part of the present disclosure.

In one embodiment, the present invention provides pharmaceutical compositions comprising a therapeutically effective amount of 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent.

In one embodiment, the present invention provides pharmaceutical compositions comprising a therapeutically effective amount of 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, at least one protective agent, and at least one pharmaceutically acceptable excipient.

In one embodiment, the present invention provides pharmaceutical compositions comprising a therapeutically effective amount of 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent.

In one embodiment, the present invention provides pharmaceutical compositions comprising a therapeutically effective amount of 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, at least one protective agent, and one at least one pharmaceutically acceptable excipient.

In one embodiment, of the pharmaceutical compositions disclosed herein comprising 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, at least 90%, or at least 95%, or at least 98%, or at least 99% of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof.

In one embodiment, the pharmaceutical compositions disclosed herein include an antioxidant as a protective agent. For example, in certain embodiments, the antioxidant can be selected from one or more of butylated hydroxyanisole, butylated hydroxytoluene, sodium metabisulfite and tert-butylhydroquinone.

In one embodiment are provided any of the pharmaceutical compositions disclosed herein wherein said antioxidant is butylated hydroxytoluene.

In one embodiment are provided any of the pharmaceutical compositions disclosed herein, wherein said at least one protective agent comprises from about 0.01% to about 99% of the total weight of said composition. In one embodiment are provided any of the pharmaceutical compositions disclosed herein, wherein said at least one protective agent comprises from about 0.01% to about 25% of the total weight of said composition. In one embodiment are provided any of the pharmaceutical compositions disclosed herein, wherein said at least one protective agent comprises from about 0.05% to about 20% of the total weight of said composition. In one embodiment are provided any of the pharmaceutical compositions disclosed herein, wherein said at least one protective agent comprises from about 0.05% to about 25% of the total weight of said composition. In one embodiment are provided any of the pharmaceutical compositions disclosed herein, wherein said at least one protective agent comprises from about 0.01% to about 15% of the total weight of said composition. In one embodiment are provided any of the pharmaceutical compositions disclosed herein, wherein said at least one protective agent comprises from about 0.05% to about 15% of the total weight of said composition. In one embodiment are provided any of the pharmaceutical compositions disclosed herein, wherein said at least one protective agent comprises from about 0.01% to about 10% of the total weight of said composition. In one embodiment are provided any of the pharmaceutical compositions disclosed herein, wherein said at least one protective agent comprises from about 0.05% to about 10% of the total weight of said composition. In one embodiment are provided any of the pharmaceutical compositions disclosed herein, wherein said at least one protective agent comprises from about 0.10% to about 10% of the total weight of said composition. In one embodiment are provided any of the pharmaceutical compositions disclosed herein, wherein said at least one protective agent comprises from about 0.10% to about 5% of the total weight of said composition. In one embodiment are provided any of the pharmaceutical compositions disclosed herein, wherein said at least one protective agent comprises from about 0.15% to about 25% of the total weight of said composition. In one embodiment are provided any of the pharmaceutical compositions disclosed herein, wherein said at least one protective agent comprises from about 0.15% to about 20% of the total weight of said composition. In one embodiment are provided any of the pharmaceutical compositions disclosed herein, wherein said at least one protective agent comprises from about 0.15% to about 15% of the total weight of said composition. In one embodiment are provided any of the pharmaceutical compositions disclosed herein, wherein said at least one protective agent comprises from about 0.15% to about 10% of the total weight of said composition. In one embodiment are provided any of the pharmaceutical compositions disclosed herein, wherein said at least one protective agent comprises from about 0.15% to about 5% of the total weight of said composition.

In one embodiment are provided any of the pharmaceutical compositions disclosed herein, wherein said at least one protective agent comprises about 0.01% of said composition. In one embodiment are provided any of the pharmaceutical compositions disclosed herein, wherein said at least one protective agent comprises about 0.05%, or about 0.1%, or about 0.25%, or about 0.50%, or about 0.75%, or about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5%, or about 5.25%, or about 5.5%, or about 5.75%, or about 6%, or about 6.25%, or about 6.5%, or about 6.75%, or about 7%, or about 7.25%, or about 7.5%, or about 7.75%, or about 8%, or about 8.25%, or about 8.5%, or about 8.75%, or about 9%, or about 9.25%, or about 9.5%, or about 9.75%, or about 10%, or about 15%, or about 25%, or about 30%, or about 40% or about 50% of the total weight of said pharmaceutical composition.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises from about 0.01% to about 25% of the total weight of said composition. In one embodiment are provided any of the pharmaceutical compositions disclosed herein wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises from about 0.25% to about 25%, or from about 0.5% to about 25%, or from about 0.75% to about 25%, or from about 1% to about 25%, or from about 0.01% to about 20%, or from about 0.1% to about 20%, or from about 0.5% to about 20%, or from about 0.5% to about 15%, or from about 0.25% to about 15%, or from about 0.5% to about 15%, or from about 0.5% to about 15%, or from about 0.75% to about 15%, or from about 1% to about 15%, or from about 1% to about 10%, or from about 1.25% to about 10%, or from about 1.5% to about 10%, or from about 1.25% to 15%, or from about 1.5% to about 10%, or from about 1.75% to about 10%, or from about 2% to about 10%, or from about 2.25% to about 15%, or from about 2.25% to about 10%, or from about 2.5% to about 15%, or from about 2.5% to about 10%, or from about 2.75% to about 15%, or from about 2.75% to about 10%, or from about 2.75% to about 5%, or from about 3% to about 15%, or from about 3% to about 10%, or from about 3% to about 7.5%, or from about 5% to about 15%, or from about 5% to 10% or from about 5% to 7.5% of the total weight of said composition.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 0.01% of the total weight of said composition. In one embodiment are provided any of the pharmaceutical compositions disclosed herein wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 0.05%, or about 0.1%, or about 0.25%, or about 0.5%, or about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5%, or about 5.25%, or about 5.5%, or about 5.75%, or about 6%, or about 6.25%, or about 6.5%, or about 6.75%, or about 7%, or about 7.25%, or about 7.5%, or about 7.75%, or about 8%, or about 8.25%, or about 8.5%, or about 8.75%, or about 9%, or about 9.25%, or about 9.5%, or about 9.75%, or about 10% of the total weight of said composition.

In one embodiment are provided any of the pharmaceutical compositions disclosed herein, wherein said composition comprises a pharmaceutically acceptable salt of 4-((1-carboxy-2-(((2E,6E)-3,7,11-trimethyldodeca-2,6,10-trien-1-yl)thio)ethyl)amino)-4-oxobutanoic acid.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises from about 0.01% to about 25% of the total weight of said composition. In one embodiment are provided any of the pharmaceutical compositions disclosed herein wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises from about 0.25% to about 25%, or from about 0.5% to about 25%, or from about 0.75% to about 25%, or from about 1% to about 25%, or from about 0.01% to about 20%, or from about 0.1% to about 20%, or from about 0.5% to about 20%, or from about 0.5% to about 15%, or from about 0.25% to about 15%, or from about 0.5% to about 15%, or from about 0.5% to about 15%, or from about 0.75% to about 15%, or from about 1% to about 15%, or from about 1% to about 10%, or from about 1.25% to about 10%, or from about 1.5% to about 10%, or from about 1.25% to 15%, or from about 1.5% to about 10%, or from about 1.75% to about 10%, or from about 2% to about 10%, or from about 2.25% to about 15%, or from about 2.25% to about 10%, or from about 2.5% to about 15%, or from about 2.5% to about 10%, or from about 2.75% to about 15%, or from about 2.75% to about 10%, or from about 2.75% to about 5%, or from about 3% to about 15%, or from about 3% to about 10%, or from about 3% to about 7.5%, or from about 5% to about 15%, or from about 5% to 10% or from about 5% to 7.5% of the total weight of said composition.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 0.01% of the total weight of said composition. In one embodiment are provided any of the pharmaceutical compositions disclosed herein wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 0.05%, or about 0.1%, or about 0.25%, or about 0.5%, or about 1%, or about 1.25%, or about 1.5%, or about 1.75%, or about 2%, or about 2.25%, or about 2.5%, or about 2.75%, or about 3%, or about 3.25%, or about 3.5%, or about 3.75%, or about 4%, or about 4.25%, or about 4.5%, or about 4.75%, or about 5%, or about 5.25%, or about 5.5%, or about 5.75%, or about 6%, or about 6.25%, or about 6.5%, or about 6.75%, or about 7%, or about 7.25%, or about 7.5%, or about 7.75%, or about 8%, or about 8.25%, or about 8.5%, or about 8.75%, or about 9%, or about 9.25%, or about 9.5%, or about 9.75%, or about 10% of the total weight of said composition.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 1% of the total weight of said composition.

In one embodiment are provided any of the pharmaceutical compositions disclosed herein, wherein said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprises about 3% of the total weight of said composition.

In one embodiment are provided any of the pharmaceutical compositions disclosed herein, wherein said composition comprises a pharmaceutically acceptable salt of 4-((1-carboxy-2-(((2E,6E)-3,7,11-trimethyldodeca-2,6,10-trien-1-yl)thio)ethyl)amino)-4-oxobutanoic acid.

In one embodiment are provided any of the pharmaceutical compositions disclosed herein, wherein at least 99% of the total amount of said pharmaceutically acceptable salt of 4-((1-carboxy-2-(((2E,6E)-3,7,11-trimethyldodeca-2,6,10-trien-1-yl)thio)ethyl)amino)-4-oxobutanoic acid comprises a pharmaceutically acceptable salt of 4-(((R)-1-carboxy-2-(((2E,6E)-3,7,11-trimethyldodeca-2,6,10-trien-1-yl)thio)ethyl)amino)-4-oxobutanoic acid.

In one embodiment are provided any of the pharmaceutical compositions disclosed herein, wherein no more than about 10% of the total amount of said pharmaceutically acceptable salt of 4-((1-carboxy-2-(((2E,6E)-3,7,11-trimethyldodeca-2,6,10-trien-1-yl)thio)ethyl)amino)-4-oxobutanoic acid, comprises a pharmaceutically acceptable salt of 4-(((S)-1-carboxy-2-(((2E,6E)-3,7,11-trimethyldodeca-2,6,10-trien-1-yl)thio)ethyl)amino)-4-oxobutanoic acid. In one embodiment are provided any of the pharmaceutical compositions disclosed herein, wherein no more than about 9%, or about 8%, or about 7%, or about 6%, or about 5%, or about 4%, or about 3%, or about 2%, or about 1%, or about 0.75%, or about 0.5%, or about 0.25% of the total amount of said pharmaceutically acceptable salt of 4-((1-carboxy-2-(((2E,6E)-3,7,11-trimethyldodeca-2,6,10-trien-1-yl)thio)ethyl)amino)-4-oxobutanoic acid, comprises a pharmaceutically acceptable salt of 4-(((S)-1-carboxy-2-(((2E,6E)-3,7,11-trimethyldodeca-2,6,10-trien-1-yl)thio)ethyl)amino)-4-oxobutanoic acid.

In one embodiment are provided any of the pharmaceutical compositions disclosed herein, wherein at least 99% of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically salt thereof, comprises a pharmaceutically acceptable salt of said 4-((1-carboxy-2-(((2E,6E)-3,7,11-trimethyldodeca-2,6,10-trien-1-yl)thio)ethyl)amino)-4-oxobutanoic acid. In one embodiment are provided any of the pharmaceutical compositions disclosed herein, wherein at least 98%, or at least 97%, or at least 96%, or at least 95%, or at least 90%, or at least 85%, or at least 80%, or at least 75%, or at least 70%, or at least 65%, or at least 60%, or at least 55%, or at least 50%, or at least 45%, or at least 40%, or at least 35%, or at least 30%, or at least 25%, or at least 20%, or at least 15%, or at least 10%, or at least 5% of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically salt thereof, comprises a pharmaceutically acceptable salt of said 4-((1-carboxy-2-(((2E,6E)-3,7,11-trimethyldodeca-2,6,10-trien-1-yl)thio)ethyl)amino)-4-oxobutanoic acid.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, a magnesium salt, a strontium salt, and a barium salt, or a mixture thereof.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, and a magnesium salt, or a mixture thereof.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, and a dipotassium salt, or a mixture thereof.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, and a disodium salt, or a mixture thereof.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is selected from a sodium salt and a disodium salt, or a mixture thereof.

In one embodiment are provided any of the pharmaceutical compositions disclosed herein, wherein said pharmaceutically acceptable salt of said 4-((1-carboxy-2-(((2E,6E)-3,7,11-trimethyldodeca-2,6,10-trien-1-yl)thio)ethyl)amino)-4-oxobutanoic acid is disodium 4-((1-carboxy-2-(((2E,6E)-3,7,11-trimethyldodeca-2,6,10-trien-1-yl)thio)ethyl)amino)-4-oxobutanoate.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, a magnesium salt, a strontium salt, and a barium salt, or a mixture thereof.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, and a magnesium salt, or a mixture thereof.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, and a dipotassium salt, or a mixture thereof.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, and a disodium salt, or a mixture thereof.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is selected from a sodium salt and a disodium salt, or a mixture thereof.

In one embodiment are provided any of the pharmaceutical compositions disclosed herein, wherein said pharmaceutically acceptable salt of said 4-(((R)-1-carboxy-2-(((2E,6E)-3,7,11-trimethyldodeca-2,6,10-trien-1-yl)thio)ethyl)amino)-4-oxobutanoic acid is disodium 4-((1-carboxy-2-(((2E,6E)-3,7,11-trimethyldodeca-2,6,10-trien-1-yl)thio)ethyl)amino)-4-oxobutanoate.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, a magnesium salt, a strontium salt, and a barium salt, or a mixture thereof.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, a dipotassium salt, a calcium salt, and a magnesium salt, or a mixture thereof.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, a disodium salt, a potassium salt, and a dipotassium salt, or a mixture thereof.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is selected from a lithium salt, a dilithium salt, a sodium salt, and a disodium salt, or a mixture thereof.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is selected from a sodium salt and a disodium salt, or a mixture thereof.

In one embodiment are provided any of the pharmaceutical compositions disclosed herein, wherein said pharmaceutically acceptable salt of said 4-(((S)-1-carboxy-2-(((2E,6E)-3,7,11-trimethyldodeca-2,6,10-trien-1-yl)thio)ethyl)amino)-4-oxobutanoic acid is disodium 4-((1-carboxy-2-(((2E,6E)-3,7,11-trimethyldodeca-2,6,10-trien-1-yl)thio)ethyl)amino)-4-oxobutanoate.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 5° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 25° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 40° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 40° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 40° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 40° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 40° C.

In one embodiment are provided any of the pharmaceutical compositions disclosed herein, wherein said pharmaceutical composition exhibits less than 20% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% degradation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 5° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 5° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 25° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 25° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 25° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 25° C.

In embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 25° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 25° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 25° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 30 days at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 60 days at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 90 days at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 6 months at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 9 months at about 40° C.

In one embodiment are provided any of the pharmaceutical compositions disclosed herein, wherein said pharmaceutical composition exhibits less than 20% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 40° C. In one embodiment are provided any of the pharmaceutical compositions disclosed herein, wherein said pharmaceutical composition exhibits less than 25%, or 30%, or 35%, or 40%, or 45%, or 50%, or 55%, or 60%, or 65%, or 70%, or 75%, or 80%, or 85%, or 90%, or 91%, or 92%, or 93%, or 94%, or 95%, or 96%, or 97%, or 98%, or 99% oxidation of the total amount of said 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, comprising said composition following storage of said composition for at least 12 months at about 40° C.

In one embodiment are provided any of the pharmaceutical compositions disclosed herein, wherein said pharmaceutical composition is suitable for topical administration to a subject.

In one embodiment are provided any of the pharmaceutical compositions disclosed herein, wherein said pharmaceutical composition is in the form of a lotion, cream, gel, spray, mist, aerosol, paste, or emulsion. In one embodiment are provided any of the pharmaceutical compositions disclosed herein, wherein said pharmaceutical composition is in the form of a lotion, cream, gel, paste or emulsion. In one embodiment are provided any of the pharmaceutical compositions disclosed herein, wherein said pharmaceutical composition is in the form of a lotion, cream, gel, or paste. In one embodiment are provided any of the pharmaceutical compositions disclosed herein, wherein said pharmaceutical composition is in the form of a lotion, cream, or gel. In one embodiment are provided any of the pharmaceutical compositions disclosed herein, wherein said pharmaceutical composition is in the form of a cream, or gel.

In one embodiment are provided any of the pharmaceutical compositions disclosed herein, wherein said pharmaceutical composition is in the form of a cream.

In one embodiment are provided any of the pharmaceutical compositions disclosed herein, wherein said pharmaceutical composition is in the form of a gel.

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least one agent wherein said agent is selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least two agents wherein said agents are selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least three agents wherein said agents are selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least four agents wherein said agents are selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least five agents wherein said agents are selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In one embodiment pharmaceutical compositions are provided, wherein said pharmaceutical composition comprises an IPC Active Agent (e.g., 4-((1-carboxy-2-(((2E,6E)-3,7,11-trimethyldodeca-2,6,10-trien-1-yl)thio)ethyl)amino)-4-oxobutanoic acid), or a pharmaceutically acceptable salt or ester thereof, and at least six agents wherein said agents are selected from the group consisting of butylated hydroxyanisole, butylated hydroxytoluene, sodium metabisulfite, tert-butylhydroquinone, propylene glycol, diethylene glycol, monoethyl ether, glycerin, methylparaben, propylparaben, benzyl alcohol, EDTA, disodium EDTA, polysorbate 80, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol (octadecenol), cholesterol, and polyoxyethylene(4)lauryl ether (e.g., Brij® 30).

In embodiments that include butylated hydroxyanisole (BHA), the butylated hydroxyanisole can be present in the pharmaceutical composition in an amount, for example, from about 0.001% to about 2% (w/w %), based on the total weight of the composition, or in an amount from about 0.005% to about 1% (e.g., 0.01%, 0.05%, 0.1%), based on the total weight of the composition.

In embodiments that include sodium metabisulfite, the sodium metabisulfite can be present in the pharmaceutical composition in an amount, for example, from about 0.01% to about 5% (w/w %), based on the total weight of the composition, or in an amount from about 0.05% to about 1% (e.g., 0.1%), based on the total weight of the composition.

In embodiments that include tert-butylhydroquinone (TBHQ), the tert-butylhydroquinone can be present in the pharmaceutical composition in an amount, for example, from about 0.001% to about 2% (w/w %), based on the total weight of the composition, or in an amount from about 0.005% to about 1% (e.g., 0.02%, 0.1%), based on the total weight of the composition.

In embodiments that include propylene glycol, the propylene glycol can be present in the pharmaceutical composition in an amount, for example, from about 0.05% to about 10% (w/w %), or from about 1% to about 10% (w/w %), based on the total weight of the composition.

In embodiments that include diethylene glycol monoethyl ether (e.g. Transcutol), the diethylene glycol monoethyl ether can be present in the pharmaceutical composition in an amount, for example, from about 0.1% to about 20% (w/w %), or from about 1% to about 10% (w/w %), based on the total weight of the composition.

In embodiments that include glycerin, the glycerin can be present in the pharmaceutical composition in an amount, for example, from about 0.1% to about 10% (w/w %), or from about 0.4% to about 10% (w/w %) based on the total weight of the composition.

In embodiments that include methylparaben, the methylparaben can be present in the pharmaceutical composition in an amount, for example, from about 0.05% to about 2% (w/w %), or from about 0.05% to about 1% (w/w %), based on the total weight of the composition.

In embodiments that include propylparaben, the propylparaben can be present in the pharmaceutical composition in an amount, for example, from about 0.01% to about 2% (w/w %), or from about 0.01% to about 0.1% (w/w %), based on the total weight of the composition.

In embodiments that include benzyl alcohol, the benzyl alcohol can be present in the pharmaceutical composition in an amount, for example, from about 0.1% to about 10% (w/w %), or from about 0.2% to about 5% (w/w %), based on the total weight of the composition.

In embodiments that include disodium EDTA, the disodium EDTA can be present in the pharmaceutical composition in an amount, for example, from about 0.01% to about 2% (w/w %), or from about 0.05% to about 0.5% (w/w %), based on the total weight of the composition.

In embodiments that include polysorbate 80, the polysorbate 80 can be present in the pharmaceutical composition in an amount, for example, from about 0.1% to about 10% (w/w %), or from about 0.1% to about 5% (w/w %), based on the total weight of the composition.

In embodiments that include hydroxyethyl cellulose, the hydroxyethyl cellulose can be present in the pharmaceutical composition in an amount, for example, from about 0.1% to about 5% (w/w %), or from about 0.12% to about 5% (w/w %), based on the total weight of the composition.

In embodiments that include emulsifying wax, the emulsifying wax can be present in the pharmaceutical composition in an amount, for example, from about 0.1% to about 30% (w/w %), or from about 2.4% to about 20% (w/w %), based on the total weight of the composition.

In embodiments that include PEG-21 stearyl ether, the PEG-21 stearyl ether can be present in the pharmaceutical composition in an amount, for example, from about 0.1% to about 10% (w/w %), or from about 0.4% to about 10% (w/w %), of from about 0.2% to about 5% (w/w %), based on the total weight of the composition.

In embodiments that include PEG-2 stearyl ether, the PEG-2 stearyl ether can be present in the pharmaceutical composition in an amount, for example, from about 0.1% to about 10% (w/w %), or from about 0.2% to about 5% (w/w %), based on the total weight of the composition.

In embodiments that include white petrolatum, the white petrolatum can be present in the pharmaceutical composition in an amount, for example, from about 0.5% to about 20% (w/w %), or from about 1% to about 20% (w/w %), based on the total weight of the composition.

In embodiments that include myristyl lactate, the myristyl lactate can be present in the pharmaceutical composition in an amount, for example, from about 0.1% to about 10% (w/w %), or from about 1% to about 10% (w/w %), based on the total weight of the composition.

In embodiments that include diisopropyl adipate, the diisopropyl adipate can be present in the pharmaceutical composition in an amount, for example, from about 0.1% to about 10% (w/w %), or from about 1% to about 10% (w/w %), based on the total weight of the composition.

In embodiments that include cetyl alcohol, the cetyl alcohol can be present in the pharmaceutical composition in an amount, for example, from about 0.1% to about 10% (w/w %), or from about 1% to about 10% (w/w %), based on the total weight of the composition.

In embodiments that include cyclomethicone, the cyclomethicone can be present in the pharmaceutical composition in an amount, for example, from about 0.1% to about 20% (w/w %), or from about 0.4% to about 20% (w/w %), based on the total weight of the composition.

In embodiments that include oleyl alcohol, the oleyl alcohol can be present in the pharmaceutical composition in an amount, for example, from about 0.1% to about 10% (w/w %), or from about 0.4% to about 10% (w/w %) based on the total weight of the composition.

In embodiments that include cholesterol, the cholesterol can be present in the pharmaceutical composition in an amount, for example, from about 0.1% to about 5% (w/w %), or from about 0.2% to about 5% (w/w %), based on the total weight of the composition.

In embodiments that include polyoxyethylene(4)lauryl ether, the polyoxyethylene(4)lauryl ether can be present in the pharmaceutical composition in an amount, for example, from about 0.01% to about 2% (w/w %), or from about 0.06% to about 1.5% (w/w %), based on the total weight of the composition.

In embodiments that include EDTA, the EDTA can be present in the pharmaceutical composition in an amount, for example, from about 0.01% to about 2% (w/w %), or from about 0.02% to about 1% (w/w %), based on the total weight of the composition.

In embodiments that include one or more poly(acrylic acid)s, the one or more poly(acrylic acid)s can individually be present in the pharmaceutical composition in an amount, for example, from about 0.1% to about 5% (w/w %), based on the total weight of the composition.

Examples of poly(acryclic acids) include, but are not limited to Carbopol 981, Permulen TRI, and Carbopol 980. In embodiments that include Carbopol 981, the Carbopol 981 can be present in the pharmaceutical composition, for example, in an amount from about 0.1% to about 5% (w/w %), or from about 0.17% to about 4.2% (w/w %), based on the total weight of the composition. In embodiments that include Permulen TRI, the Permulen TRI can be present in the pharmaceutical composition, for example, in an amount from about 0.1% to about 5% (w/w %), or from about 0.1% to about 1% (w/w %), based on the total weight of the composition. In embodiments that include Carbopol 980, the Carbopol 980 can be present in the pharmaceutical composition, for example, in an amount from about 0.1% to about 5% (w/w %), or from about 0.1% to about 3% (w/w %), based on the total weight of the composition.

In one embodiment, a pharmaceutical composition comprises from about 0.01% to about 25% of IPC Active Agent, from about 0.1% to about 20% of diethylene glycol monoethyl ether (e.g. Transcutol), from about 0.1% to about 10% of glycerin, from about 0.05% to about 2% of methylparaben, from about 0.01% to about 2% of propylparaben, from about 0.01% to about 2% of disodium EDTA, from about 0.001% to about 2% of butylated hydroxyanisole, from about from about 0.1% to about 10% of polysorbate 80 and from about 0.1% to about 5% of hydroxyethyl cellulose, all based on the total weight of the composition.

In one embodiment, a pharmaceutical composition comprises from about 0.1% to about 10% of PEG-21 stearyl ether (e.g. Brij 721), from about 0.1% to about 10% of PEG-2 stearyl ether (e.g., Brij 72), from about 0.5% to about 20% of white petrolatum, from about 0.1% to about 10% of diisopropyl adipate, from about 0.1% to about 10% of cetyl alcohol, from about 0.1% to about 20% of cyclomethicone, from about 0.1% to about 10% of oleyl alcohol, from about 0.001% to about 2% of butylated hydroxytoluene, from about 0.001% to about 2% of butylated hydroxyanisole, from about 0.01% to about 25% of IPC Active Agent, from about 0.5% to about 10% of propylene glycol, from about 0.05% to about 2% of methylparaben, from about 0.01% to about 2% of propylparaben, from about 0.01% to about 2% of EDTA, and from about 0.1% to about 5% of poly(acrylic acid) (e.g. Carbopol 980).

In one embodiment pharmaceutical compositions are provided, wherein said pharmaceutical composition comprises a therapeutically effective amount of 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent wherein said protective agent is butylated hydroxytoluene.

In one embodiment are pharmaceutical compositions, wherein said pharmaceutical composition comprises a therapeutically effective amount of 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent wherein said protective agent is tert-butyl hydroquinone.

In one embodiment are pharmaceutical compositions, wherein said pharmaceutical composition comprises a therapeutically effective amount of 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent wherein said protective agent is butylated hydroxytoluene.

In one embodiment are pharmaceutical compositions, wherein said pharmaceutical composition comprises a therapeutically effective amount of 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent wherein said protective agent is tert-butyl hydroquinone.

In one embodiment is provided a method of treating a skin disorder in a subject, comprising administering to said subject a therapeutically effective amount of any of the pharmaceutical compositions disclosed herein, and wherein said skin disorder is selected from acne, atopic dermatitis, and rosacea. In one embodiment, the skin disorder is acne. In embodiment, the skin disorder is atopic dermatitis. In one embodiment, the skin disorder is rosacea.

In one embodiment is provided a method of treating inflammatory lesions associated with rosacea in a subject, comprising administering to said subject a therapeutically effective amount of any of the pharmaceutical compositions disclosed herein to the areas of the skin of said subject affected by said inflammatory lesions associated with rosacea.

In one embodiment is provided a method of treating persistent facial erythema of rosacea in a subject, comprising administering to said subject a therapeutically effective amount of any of the pharmaceutical compositions disclosed herein to the areas of the skin of said subject affected by said facial erythema.

In one embodiment is provided a method of treating papulopustular rosacea in a subject, comprising administering to said subject a therapeutically effective amount of any of the pharmaceutical compositions disclosed herein to the areas of the skin of said subject affected by said papulopustual rosacea.

In one embodiment is provided a method of treating inflammatory lesions of rosacea in a subject, comprising administering to said subject a therapeutically effective amount of any of the pharmaceutical compositions disclosed herein to the areas of the skin of said subject affected by said inflammatory lesions of rosacea.

In one embodiment is provided a method of treating redness associated with rosacea in a subject, comprising administering to said subject a therapeutically effective amount of any of the pharmaceutical compositions disclosed herein to the areas of the skin of said subject affected by said redness associated with rosacea in a subject.

In one embodiment is provided a method of treating or preventing a skin disorder in a subject, comprising administering to said subject a therapeutically effective amount of any of the pharmaceutical compositions disclosed herein, wherein said skin disorder is selected from rosacea, erythema of rosacea, and erythema of acne.

In one embodiment is provided a method of treating inflammatory papules and pustules of mild to moderate rosacea in subject, comprising administering to said subject a therapeutically effective amount of any of the pharmaceutical compositions disclosed herein to the areas of the skin of said subject affected by said inflammatory papules and pustules of mild to moderate rosacea.

In one embodiment is provided a method of treating acne vulgaris in a subject, comprising administering to said subject a therapeutically effective amount of any of the pharmaceutical compositions disclosed herein to the areas of the skin of said subject affected by said acne vulgaris.

In one embodiment is provided a method of treating inflammatory acne vulgaris in a subject, comprising administering to said subject a therapeutically effective amount of any of the pharmaceutical compositions disclosed herein to the areas of the skin of said subject affected by said inflammatory acne vulgaris.

In one embodiment is provided a method of treating any of the skin conditions in a subject disclosed herein, wherein said pharmaceutical composition is administered to said subject once daily, twice daily, three times daily, four times daily, or five times daily.

In one embodiment is provided a method of treating any of the skin conditions in a subject disclosed herein, wherein said pharmaceutical composition is administered to said subject once in the morning and once in the evening.

In one embodiment is provided a method of treating any of the skin conditions in a subject disclosed herein, wherein said pharmaceutical composition is administered to said subject from one week to 12 months. In one embodiment is provided a method of treating any of the skin conditions in a subject disclosed herein, wherein said pharmaceutical composition is administered to said subject from 2 weeks to 12 months, or from 3 weeks to 12 months, or from 4 weeks to 12 months, or from 5 weeks to 12 months, or from one week to 9 months, or from one week to 6 months, or from 2 weeks to 9 months, or from 3 weeks to 9 months, or from 4 weeks to 9 months, or from 4 weeks to 6 months.

In one embodiment is provided a method of treating any of the skin conditions in a subject disclosed herein, wherein said pharmaceutical composition is administered to said subject for one week. In one embodiment is provided a method of treating any of the skin conditions in a subject disclosed herein, wherein said pharmaceutical composition is administered to said subject for 2 weeks, or for 3 weeks, or for 4 weeks, or for 5 weeks, or for 6 weeks, or for 7 weeks, or for 8 weeks, or for 3 months, or for 4 months, or for 5 months, or for 6 months, or for 7 months, or for 8 months, or for 9 months, or for 10 months, or for 11 months, or for 12 months.

In one embodiment is provided a method of treating any of the skin conditions in a subject disclosed herein, wherein said pharmaceutical composition is administered to each of the five areas of the face of said subject.

In one embodiment are provided any of the pharmaceutical compositions disclosed herein for use in the treatment of a skin disorder in a subject, comprising administering to said subject a therapeutically effective amount of said pharmaceutical composition, wherein said skin disorder is selected from acne, atopic dermatitis, and rosacea. In another embodiment, the skin disorder is acne. In another embodiment, the skin disorder is atopic dermatitis. In another embodiment, the skin disorder is rosacea.

In one embodiment are provided any of the pharmaceutical compositions disclosed herein for use in in the treatment of inflammatory lesions associated with rosacea in a subject, comprising administering to said subject a therapeutically effective amount of said pharmaceutical composition to the areas of the skin of said subject affected by said inflammatory lesions associated with rosacea.

In one embodiment are provided any of the pharmaceutical compositions disclosed herein for use in for the treatment of persistent facial erythema of rosacea in a subject, comprising administering to said subject a therapeutically effective amount of said pharmaceutical composition to the areas of the skin of said subject affected by said facial erythema.

In one embodiment are provided any of the pharmaceutical compositions disclosed herein for use in the treatment of papulopustular rosacea in a subject, comprising administering to said subject a therapeutically effective amount of said pharmaceutical composition to the areas of the skin of said subject affected by said papulopustual rosacea.

In one embodiment are provided any of the pharmaceutical compositions disclosed herein for use in the treatment of inflammatory lesions of rosacea in a subject, comprising administering to said subject a therapeutically effective amount of said pharmaceutical composition to the areas of the skin of said subject affected by said inflammatory lesions of rosacea.

In one embodiment are provided any of the pharmaceutical compositions disclosed herein for use in the treatment of redness associated with rosacea in a subject, comprising administering to said subject a therapeutically effective amount of said pharmaceutical composition to the areas of the skin of said subject affected by said redness associated with rosacea in a subject.

In one embodiment are provided any of the pharmaceutical compositions disclosed herein for use in the treatment or prevention of a skin disorder in a subject, comprising administering to said subject a therapeutically effective amount of said pharmaceutical composition, wherein said skin disorder is selected from rosacea, erythema of rosacea, and erythema of acne.

In one embodiment are provided any of the pharmaceutical compositions disclosed herein for use in the treatment of inflammatory papules and pustules of mild to moderate rosacea in subject, comprising administering to said subject a therapeutically effective amount of said pharmaceutical composition to the areas of the skin of said subject affected by said inflammatory papules and pustules of mild to moderate rosacea.

In one embodiment are provided any of the pharmaceutical compositions disclosed herein for use in the treatment of acne vulgaris in a subject, comprising administering to said subject a therapeutically effective amount of said pharmaceutical composition to the areas of the skin of said subject affected by said acne vulgaris.

In one embodiment are provided any of the pharmaceutical compositions disclosed herein for use in the treatment of inflammatory acne vulgaris in a subject, comprising administering to said subject a therapeutically effective amount of said pharmaceutical composition to the areas of the skin of said subject affected by said inflammatory acne vulgaris.

In one embodiment are provided any of uses of the pharmaceutical compositions disclosed herein, wherein said pharmaceutical composition is administered to said subject once daily, twice daily, three times daily, four times daily, or five times daily.

In one embodiment are provided any of the uses of the pharmaceutical compositions disclosed herein, wherein said pharmaceutical composition is administered to said subject once in the morning and once in the evening.

In one embodiment are provided any of the uses of the pharmaceutical compositions disclosed herein, wherein said pharmaceutical composition is administered to said subject from one week to 12 months.

In one embodiment are provided any of the uses of the pharmaceutical compositions disclosed herein, wherein said pharmaceutical composition is administered to each of the five areas of the face of said subject.

In one embodiment is provided a kit, comprising any of the pharmaceutical compositions disclosed herein and printed instructions for use of said pharmaceutical composition.

Use any of the pharmaceutical compositions disclosed herein, for the manufacture of a medicament for the treatment of a skin disorder in a subject, wherein said skin disorder is selected from acne, atopic dermatitis, and rosacea. In one embodiment, the skin condition is acne. In one embodiment, the skin condition is atopic dermatitis. In one embodiment, the skin condition is rosacea.

Use any of the pharmaceutical compositions disclosed herein for the manufacture of a medicament for the treatment of inflammatory lesions associated with rosacea in a subject.

Use any of the pharmaceutical compositions disclosed herein for the manufacture of a medicament for the treatment of persistent facial erythema of rosacea in a subject.

Use any of the pharmaceutical compositions disclosed herein for the manufacture of a medicament for the treatment of papulopustular rosacea in a subject.

Use any of the pharmaceutical compositions disclosed herein for the manufacture of a medicament for the treatment of inflammatory lesions of rosacea in a subject.

Use any of the pharmaceutical compositions disclosed herein for the manufacture of a medicament for the treatment of redness associated with rosacea in a subject.

Use any of the pharmaceutical compositions disclosed herein for the manufacture of a medicament for the treatment or prevention of a skin disorder in a subject, wherein said skin disorder is selected from rosacea, erythema of rosacea, and erythema of acne.

Use any of the pharmaceutical compositions disclosed herein for the manufacture of medicament for the treatment of inflammatory papules and pustules of mild to moderate rosacea in subject.

Use any of the pharmaceutical compositions disclosed herein for the manufacture of a medicament for the treatment of acne vulgaris in a subject.

Use any of the pharmaceutical compositions disclosed herein for the manufacture of a medicament for the treatment of inflammatory acne vulgaris in a subject.

Any of the uses disclosed herein, wherein said pharmaceutical composition is administered to said subject once daily, twice daily, three times daily, four times daily, or five times daily.

Any of the uses disclosed herein, wherein said pharmaceutical composition is administered to said subject once in the morning and once in the evening.

Any of the uses disclosed herein, wherein said pharmaceutical composition is administered to said subject from one week to 12 months. Any of the uses disclosed herein wherein said pharmaceutical composition is administered to said subject from 2 weeks to 12 months, or from 3 weeks to 12 months, or from 4 weeks to 12 months, or from 5 weeks to 12 months, or from one week to 9 months, or from one week to 6 months, or from 2 weeks to 9 months, or from 3 weeks to 9 months, or from 4 weeks to 9 months, or from 4 weeks to 6 months.

Any of the uses disclosed herein wherein said pharmaceutical composition is administered to said subject for one week. Any of the uses disclosed herein, wherein said pharmaceutical composition is administered to said subject for 2 weeks, or for 3 weeks, or for 4 weeks, or for 5 weeks, or for 6 weeks, or for 7 weeks, or for 8 weeks, or for 3 months, or for 4 months, or for 5 months, or for 6 months, or for 7 months, or for 8 months, or for 9 months, or for 10 months, or for 11 months, or for 12 months

Any of the uses disclosed herein, wherein said pharmaceutical composition is administered to each of the five areas of the face of said subject.

Use any of the pharmaceutical compositions disclosed herein in the manufacture of a medicament substantially as herein described and illustrated.

A pharmaceutical composition substantially as hereinbefore described with reference to any one of the examples.

A method of treatment substantially as hereinbefore described with reference to any one of the examples.

Use of a pharmaceutical composition substantially as hereinbefore described with reference to any one of the examples.

In one embodiment, the compositions disclosed herein comprise 4-((1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent, wherein the at least one protective agent comprises an antioxidant. In one embodiment, the compositions disclosed herein comprise a pharmaceutically acceptable salt of 4-((1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant. In one embodiment, the compositions disclosed herein comprise the disodium salt of 4-((1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant.

In one embodiment, the compositions disclosed herein comprise 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent, wherein the at least one protective agent comprises an antioxidant. In one embodiment, the compositions disclosed herein comprise a pharmaceutically acceptable salt of 4-(((R)-1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant. In one embodiment, the compositions disclosed herein comprise the disodium salt of 4-(((R)-1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant.

In one embodiment, the compositions disclosed herein comprise 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof, and at least one protective agent, wherein the protective agent comprises an antioxidant. In one embodiment, the compositions disclosed herein comprise a pharmaceutically acceptable salt of 4-(((S)-1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant. In one embodiment, the compositions disclosed herein comprise the disodium salt of 4-(((S)-1-carboxy-2-(((2E,6E)-3,7,11-trimethyldodeca-2,6,10-trien-1-yl)thio)ethyl)amino)-4-oxobutanoic acid, and at least one protective agent, wherein the at least one protective agent comprises an antioxidant.

As used herein, the term “therapeutically effective amount” means that amount of the compound or compounds being administered which will relieve to some extent one or more of the symptoms of the disorder being treated. In reference to the treatment of a skin condition selected from acne, atopic dermatitis, and rosacea, a therapeutically effective amount of a pharmaceutical compositions as disclosed herein means that amount such pharmaceutical composition which has one or more of the following effects in a subject to which such pharmaceutical compositions are administered of (1) reducing or preventing redness or erythema associated with such conditions, (2) reducing or preventing the amount of inflammation associated with such conditions, (3) reducing or preventing inflammatory lesions associated with such conditions, (4) reducing or preventing papulopustular rosacea, or (5) reducing or preventing inflammatory papules and pustules associated with such conditions.

In addition to the above, as known to those skilled in the art, the compounds disclosed herein may be present as a mixture of tautomers. Unless otherwise noted herein, the depiction of the chemical structures of the compounds disclosed herein are meant to encompass each such tautomeric form and mixtures of the tautomeric forms.

Unless indicated otherwise, all references herein to compounds disclosed herein, or a pharmaceutically acceptable salt thereof, include references to salts, solvates, hydrates and complexes thereof, and to solvates, hydrates and complexes of salts thereof, including polymorphs, stereoisomers, and isotopically labeled versions thereof.

The compounds disclosed herein may exist in the form of pharmaceutically acceptable salts such as, e.g., acid addition salts and base addition salts of the compounds of one of the formulae provided herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein.

For example, the compounds 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, and 4-(((S)-1-carboxy-2-(((2E,6E)-3,7,11-trimethyldodeca-2,6,10-trien-1-yl)thio)ethyl)amino)-4-oxobutanoic acid are capable of forming salts with various pharmacologically acceptable cations or counterions. Examples of such salts include the alkali metal or alkaline-earth metal salts and particularly, the sodium and potassium salts. These salts may be prepared by conventional techniques. The chemical bases which are used as reagents to prepare the pharmaceutically acceptable base salts of the compounds are those which form non-toxic base salts with the compounds herein. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

In one embodiment, the compositions described herein comprise 4-((1-carboxy-2-(((2E,6E)-3,7,11-trimethyldodeca-2,6,10-trien-1-yl)thio)ethyl)amino)-4-oxobutanoic acid. In one embodiment, the compositions described herein comprise 4-(((R)-1-carboxy-2-(((2E,6E)-3,7,11-trimethyldodeca-2,6,10-trien-1-yl)thio)ethyl)amino)-4-oxobutanoic acid. In one embodiment, the compositions described herein comprise 4-(((S)-1-carboxy-2-(((2E,6E)-3,7,11-trimethyldodeca-2,6,10-trien-1-yl)thio)ethyl)amino)-4-oxobutanoic acid.

In one embodiment, the compositions described herein comprise a pharmaceutically acceptable salt of 4-((1-carboxy-2-(((2E,6E)-3,7,11-trimethyldodeca-2,6,10-trien-1-yl)thio)ethyl)amino)-4-oxobutanoic acid. In one embodiment, the compositions described herein comprise a pharmaceutically acceptable salt of 4-(((R)-1-carboxy-2-(((2E,6E)-3,7,11-trimethyldodeca-2,6,10-trien-1-yl)thio)ethyl)amino)-4-oxobutanoic acid. In one embodiment, the compositions described herein comprise a pharmaceutically acceptable salt of 4-(((S)-1-carboxy-2-(((2E,6E)-3,7,11-trimethyldodeca-2,6,10-trien-1-yl)thio)ethyl)amino)-4-oxobutanoic acid.

In one embodiment, the compositions described herein comprise the disodium salt of 4-((1-carboxy-2-(((2E,6E)-3,7,11-trimethyldodeca-2,6,10-trien-1-yl)thio)ethyl)amino)-4-oxobutanoic acid. In one embodiment, the compositions described herein comprise the disodium salt of 4-(((R)-1-carboxy-2-(((2E,6E)-3,7,11-trimethyldodeca-2,6,10-trien-1-yl)thio)ethyl)amino)-4-oxobutanoic acid. In one embodiment, the compositions described herein comprise the disodium salt of 4-(((S)-1-carboxy-2-(((2E,6E)-3,7,11-trimethyldodeca-2,6,10-trien-1-yl)thio)ethyl)amino)-4-oxobutanoic acid.

The chemical bases that may be used as reagents to prepare pharmaceutically acceptable base salts of the compounds disclosed herein are those that form non-toxic base salts with such compounds. Such non-toxic base salts include, but are not limited to, those derived from such pharmacologically acceptable cations such as alkali metal cations (e.g., lithium, sodium and potassium) and alkaline earth metal cations (e.g., calcium, magnesium, strontium and barium), ammonium or water-soluble amine addition salts such as N-methylglucamine-(meglumine), and the lower alkanolammonium and other base salts of pharmaceutically acceptable organic amines. Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

Salts of the compounds disclosed herein can be prepared according to methods known to those of skill in the art. A pharmaceutically acceptable salt of the inventive compounds can be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Pharmaceutically acceptable salts of the compounds disclosed herein may be prepared by one or more of the following methods: (i) by reacting the compound disclosed herein with the desired base; (ii) by removing an acid- or base-labile protecting group from a suitable precursor of the compound disclosed herein or by ring-opening a suitable cyclic precursor, for example, a lactone or lactam, using the desired base; or (iii) by converting one salt of the compound disclosed herein to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds disclosed herein may exist in both unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm. The term “solvate” is used herein to describe a molecular complex comprising the compound disclosed herein and one or more pharmaceutically acceptable solvent molecules, for example, ethanol. The term “hydrate” is used when the solvent is water. Pharmaceutically acceptable solvates in accordance with the embodiments disclosed herein include hydrates and solvates wherein the solvent of crystallization may be isotopically substituted, e.g. D₂O, d6-acetone, d6-DMSO.

Also included within the scope disclosed herein are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see Haleblian, J. Pharm. Sci., 1975, 64 (8): 1269-1288, the disclosure of which is incorporated herein by reference in its entirety.

The compounds disclosed herein include all polymorphs and crystal habits thereof, prodrugs and isomers thereof (including optical, geometric and tautomeric isomers) as hereinafter defined and isotopically-labeled compounds disclosed herein.

The compounds disclosed herein have asymmetric carbon atoms. The carbon-carbon bonds of the compounds disclosed herein may be depicted herein using a solid line, a solid wedge, or a dotted wedge. The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds disclosed herein may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds disclosed herein can exist as enantiomers or as racemates and mixtures thereof.

Compounds disclosed herein can exist as stereoisomers, such as racemates, or the (R)- or (S)-stereoisomer. Stereoisomers of the compounds of the formulae herein can include cis and trans isomers, optical isomers such as (R) and (S) enantiomers, diastereomers, geometric isomers, rotational isomers, atropisomers, conformational isomers, and tautomers of the compounds disclosed herein, including compounds exhibiting more than one type of isomerism; and mixtures thereof (such as racemates and diastereomeric pairs). Also included are acid addition or base addition salts wherein the counterion is optically active, for example, d-lactate or l-lysine, or racemic, for example, dl-tartrate or dl-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds disclosed herein may exhibit the phenomena of tautomerism and structural isomerism. For example, the compounds may exist in several tautomeric forms, including the enol and imine form, and the keto and enamine form and geometric isomers and mixtures thereof. All such tautomeric forms are included within the scope of compounds disclosed herein. Tautomers exist as mixtures of a tautomeric set in solution. In solid form, usually one tautomer predominates. Even though one tautomer may be described, the compounds disclosed herein are meant to encompass all tautomers of the compounds of the formulae provided.

In addition, some of the compounds disclosed herein may form atropisomers (e.g., substituted biaryls). Atropisomers are conformational stereoisomers which occur when rotation about a single bond in the molecule is prevented, or greatly slowed, as a result of steric interactions with other parts of the molecule and the substituents at both ends of the single bond are unsymmetrical. The interconversion of atropisomers is slow enough to allow separation and isolation under predetermined conditions. The energy barrier to thermal racemization may be determined by the steric hindrance to free rotation of one or more bonds forming a chiral axis.

Where one or more compounds disclosed herein contains an alkenyl or alkenylene group, geometric cis/trans (or Z/E) isomers are possible. Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art.

Chiral compounds disclosed herein (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art; see, for example, “Stereochemistry of Organic Compounds” by E L Eliel (Wiley, New York, 1994), the disclosure of which is incorporated herein by reference in its entirety.

As used herein, the term “enantiomerically pure” describes one or more compounds that is present as a single enantiomer and which is described in terms of enantiomeric excess (e.e.). Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, “diastereomerically pure” as used herein, describes one or more compounds that is present as a diastereomer and which is described in terms of diasteriomeric excess (d.e.). Preferably, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

In another embodiment are included isotopically-labeled compounds, which are identical to those recited in one of the formulae provided, but for the fact that one or more atoms are replaced by an atom having an atomic mass or mass number different from the atomic mass or mass number usually found in nature.

Isotopically-labeled compounds disclosed herein can generally be prepared by conventional techniques known to those skilled in the art or by processes analogous to those described herein, using an appropriate isotopically-labeled reagent in place of the non-labeled reagent otherwise employed.

Examples of isotopes that may be incorporated into compounds disclosed herein include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl. Certain isotopically-labeled compounds disclosed herein, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds disclosed herein may generally be prepared by carrying out the procedures disclosed in the Schemes and/or in the Examples and Preparations below, by substituting an isotopically-labeled reagent for a non-isotopically-labeled reagent. Pharmaceutically acceptable solvates in accordance with the present disclosure include those wherein the solvent of crystallization may be isotopically substituted, e.g. D₂O, d⁶-acetone, d⁶-DMSO.

The compounds disclosed herein intended for pharmaceutical use may be administered as crystalline or amorphous products, or mixtures thereof. They may be obtained, for example, as solid plugs, powders, or films by methods such as precipitation, crystallization, freeze drying, spray drying, or evaporative drying. Microwave or radio frequency drying may be used for this purpose.

Some embodiments relate to compositions comprising one or more compounds disclosed herein, or a pharmaceutically acceptable salt or ester thereof (e.g., pharmaceutical compositions). In another embodiment are provided pharmaceutical compositions comprising one or more compounds disclosed herein, or a pharmaceutically acceptable salt or ester thereof, one or more pharmaceutically acceptable carriers and, optionally, at least one additional medicinal or pharmaceutical agent. In some embodiments, the at least one additional medicinal or pharmaceutical agent is an anti-cancer agent as described below.

The pharmaceutically acceptable carrier may comprise a conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages.

In some embodiments, the composition comprises a therapeutically effective amount of one or more compounds disclosed herein, or a pharmaceutically acceptable salt or ester thereof, and one or more pharmaceutically acceptable carriers.

The compounds disclosed herein, or their pharmaceutically acceptable salts or esters, may be formulated into pharmaceutical compositions as described below in any pharmaceutical form recognizable to the skilled artisan as being suitable. Pharmaceutical compositions disclosed herein comprise a therapeutically effective amount of at least one compound disclosed herein and an inert, pharmaceutically acceptable carrier or diluent.

To treat or prevent conditions described herein, a pharmaceutical composition as disclosed herein is administered in a suitable formulation prepared by combining a therapeutically effective amount of an IPC Active Agent (e.g., 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, or 4-(((S)-1-carboxy-2-(((2E,6E)-3,7,11-trimethyldodeca-2,6,10-trien-1-yl)thio)ethyl)amino)-4-oxobutanoic acid), a pharmaceutically acceptable salt or ester thereof, or a mixture thereof, as the case may be, with one or more pharmaceutically suitable carriers, which may be selected, for example, from diluents, excipients and auxiliaries that facilitate processing of the active compounds into the final pharmaceutical preparations.

The pharmaceutical carriers employed may be either solid or liquid. Exemplary solid carriers are lactose, sucrose, talc, gelatin, agar, pectin, acacia, magnesium stearate, stearic acid and the like. Exemplary liquid carriers are syrup, peanut oil, olive oil, water and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire®, a semi-solid vehicle that protects active ingredients from light, moisture and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, one or more compounds disclosed herein, or a pharmaceutically acceptable salt or ester thereof, may be dissolved in an aqueous solution of an organic or inorganic acid, such as a 0.3 M solution of succinic acid or citric acid. If a soluble salt form is not available, the compound or salt may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, one or more compounds disclosed herein, or a pharmaceutically acceptable salt or ester thereof, is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds disclosed herein, or a pharmaceutically acceptable salt or ester thereof, may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds disclosed herein, or a pharmaceutically acceptable salt or ester thereof, can be formulated by combining the compound with pharmaceutically acceptable carriers known in the art. Such carriers enable the compounds disclosed herein to be formulated as tablets, pills, dragees, capsules, liquids, gels, syrups, slurries, suspensions and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present disclosure may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds disclosed herein, or a pharmaceutically acceptable salt or ester thereof, may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the compounds disclosed herein, or a pharmaceutically acceptable salt or ester thereof, may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the compounds disclosed herein, or a pharmaceutically acceptable salt or ester thereof, may be in powder form for constitution with a suitable vehicle, e.g. sterile pyrogen-free water, before use.

In addition to the formulations described above, the compounds disclosed herein, or a pharmaceutically acceptable salt or ester thereof, may also be formulated as a depot preparation. Such long-acting formulations may be administered by implantation (for example, subcutaneously or intramuscularly) or by intramuscular injection. Thus, for example, the compounds disclosed herein, or a pharmaceutically acceptable salt or ester thereof, may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5 W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions disclosed herein may also comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols. Furthermore, additives or excipients such as Gelucire®, Capryol®, Labrafil®, Labrasol®, Lauroglycol®, Plurol®, Peceol®, Transcutol® and the like may be used.

Appropriate excipients are selected based on the active agent and the type of the formulation. Standard excipients include, but are not limited to, gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethyl cellulose calcium, carboxymethyl cellulose sodium, methylcellulose, hydroxyethyl cellulose, hydroxypropyl cellulose, hydroxypropylmethyl cellulose phthalate, noncrystalline cellulose, magnesium aluminum silicate, triethanolamine, polyvinyl alcohol, polyvinylpyrrolidone, sugars, and starches.

In some embodiments, the composition or formulation further comprises one or more emollients. Emollients are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof.

In some embodiments, the composition or formulation further comprises one or more buffers. In some embodiments, the one or more buffers maintain the composition or formulation at a pH of from about 4 to about 7.5. In some embodiments, the one or more buffers maintain the composition or formulation at a pH of from about 4 to about 7. In some embodiments, the one or more buffers maintain the composition or formulation at a pH of from about 5 to about 7.

In some embodiments, the composition or formulation further comprises one or more penetration enhancers. Penetration enhancers are frequently used to promote transdermal delivery of drugs across the skin, in particular across the stratum corneum. Some penetration enhancers cause dermal irritation, dermal toxicity and dermal allergies. However, the more commonly used ones include, but are not limited to, dimethyl sulfoxide, urea, (carbonyldiamide), imidurea, N,N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). In some embodiments, the one or more penetration enhancer comprises dimethyl sulfoxide.

In some embodiments, the formulation is a gel. A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. In some embodiments, the one or more gelling agents are natural, semi-synthetic, or synthetic. Suitable thickening or gelling agents include, but are not limited to, acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C₉-C₁₅ alcohols, calcium acetate, calcium alginate, calcium carrageenan, calcium chloride, caprylic alcohol, vinyl polymers such as cross linked acrylic acid polymers with the name carbomer, such as but not limited to carbomer 910, carbomer 934, carbomer 934P, carbomer 940, carbomer 941; modified celluloses such as hydroxypropyl cellulose and hydroxyethyl cellulose; Carbopol homopolymers and copolymers, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M is also known as Polyox WSR® N-IO, which is available from Union Carbide and as PEG-2,000; PEG-5M is also known as Polyox WSR® N-35 and Polyox WSR® N-80, both available from Union Carbide and as PEG-5,000 and Polyethylene Glycol 300,000; PEG-7M is also known as Polyox WSR® N-750 available from Union Carbide; PEG 9-M is also known as Polyox WSR® N-3333 available from Union Carbide; PEG-14M is also known as Polyox WSR® N-3000 available from Union Carbide, polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacrylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof. In some embodiments, the one or more gelling agents comprise hydroxypropyl cellulose (HPC).

The concentration of one or more gelling agents can be adjusted to change the viscosity of the gel. For example, in some embodiments the formulation includes less than 1%, or about 1%, 2%, 3%, 4%, 5%, 6%, 7%, 8%, 9%, 10%, 20%, 30%, 40%, 50%, 60%, 70%, or 80% w/w, including increments therein, of the one or more gelling agents. In some embodiments, the one or more gelling agents are present at from about 0.1% w/w to about 80% w/w. In some embodiments, the one or more gelling agents are present at from about 0.5% w/w to about 10% w/w. In some embodiments, the one or more gelling agents are present at from about 0.5% w/w to about 5% w/w. In some embodiments, the one or more gelling agents are present at from about 1% w/w to about 3% w/w.

In some embodiments, the composition or formulation has a viscosity of at least 100 cP. In some embodiments, the composition or formulation has a viscosity of at least 500 cP. In some embodiments, the composition or formulation has a viscosity of at least 1,000 cP, or at least 2,000 cP, or at least 3,000 cP, or at least 4,000 cP, or at least 5,000 cP, or at least 6,000 cP, or at least 7,000 cP, or at least 8,000 cP, or at least 9,000 cP, or at least 10,000 cP, or at least 11,000 cP, or at least 12,000 cP, or at least 13,000 cP, or at least 14,000 cP, or at least 15,000 cP, or at least 16,000 cP, or at least 17,000 cP, or at least 18,000 cP, or at least 19,000 cP, or at least 20,000 cP. In some embodiments, the composition or formulation has a viscosity of at least 5000 cP.

In some embodiments, the composition or formulation has a viscosity of not less than 500 cP, or not less than 1000 cP, or not less than 1500 cP, or not less than 2000 cP, or not less than 2500 cP, or not less than 3000 cP, or not less than 3500 cP, or not less than 4000 cP, or not less than 4500 cP, or not less than 5000 cP, or not less than 5500 cP, or not less than 6000 cP, or not less than 7000 cP, or not less than 8000 cP, or not less than 9000 cP, or not less than 9000 cP, or not less than 10,000 cP, or not less than 11,000 cP, or not less than 12,000 cP, or not less than 13,000 cP, or not less than 14,000 cP, or not less than 15,000 cP, or not less than 16,000 cP, or not less than 17,000 cP, or not less than 18,000 cP, or not less than 19,000 cP, or not less than 20,000 cP.

In some embodiments, the composition or formulation has a viscosity of about 100 cP to about 20,000 cP. In some embodiments, the composition or formulation has a viscosity of about 100 cP to about 20,000 cP. In some embodiments, the composition or formulation has a viscosity of about 200 cP to about 20,000 cP. In some embodiments, the composition or formulation has a viscosity of about 300 cP to about 20,000 cP. In some embodiments, the composition or formulation has a viscosity of about 400 cP to about 20,000 cP. In some embodiments, the composition or formulation has a viscosity of about 500 cP to about 20,000 cP. In some embodiments, the composition or formulation has a viscosity of about 600 cP to about 20,000 cP. In some embodiments, the composition or formulation has a viscosity of about 700 cP to about 20,000 cP. In some embodiments, the composition or formulation has a viscosity of about 800 cP to about 20,000 cP. In some embodiments, the composition or formulation has a viscosity of about 900 cP to about 20,000 cP. In some embodiments, the composition or formulation has a viscosity of about 1000 cP to about 20,000 cP. In some embodiments, the composition or formulation has a viscosity of about 2000 cP to about 20,000 cP. In some embodiments, the composition or formulation has a viscosity of about 3000 cP to about 20,000 cP. In some embodiments, the composition or formulation has a viscosity of about 4000 cP to about 20,000 cP. In some embodiments, the composition or formulation has a viscosity of about 5000 cP to about 20,000 cP. In some embodiments, the composition or formulation has a viscosity of about 6000 cP to about 20,000 cP. In some embodiments, the composition or formulation has a viscosity of about 7000 cP to about 20,000 cP. In some embodiments, the composition or formulation has a viscosity of about 8000 cP to about 20,000 cP. In some embodiments, the composition or formulation has a viscosity of about 9000 cP to about 20,000 cP. In some embodiments, the composition or formulation has a viscosity of about 1000 cP to about 20,000 cP.

In some embodiments, the composition or formulation has a viscosity of about 1000 cP to about 17,000 cP. In some embodiments, the composition or formulation has a viscosity of about 100 cP to about 17,000 cP. In some embodiments, the composition or formulation has a viscosity of about 200 cP to about 17,000 cP. In some embodiments, the composition or formulation has a viscosity of about 300 cP to about 17,000 cP. In some embodiments, the composition or formulation has a viscosity of about 400 cP to about 17,000 cP. In some embodiments, the composition or formulation has a viscosity of about 500 cP to about 17,000 cP. In some embodiments, the composition or formulation has a viscosity of about 600 cP to about 17,000 cP. In some embodiments, the composition or formulation has a viscosity of about 700 cP to about 17,000 cP. In some embodiments, the composition or formulation has a viscosity of about 800 cP to about 17,000 cP. In some embodiments, the composition or formulation has a viscosity of about 900 cP to about 17,000 cP. In some embodiments, the composition or formulation has a viscosity of about 1000 cP to about 17,000 cP. In some embodiments, the composition or formulation has a viscosity of about 2000 cP to about 17,000 cP. In some embodiments, the composition or formulation has a viscosity of about 3000 cP to about 17,000 cP. In some embodiments, the composition or formulation has a viscosity of about 4000 cP to about 17,000 cP. In some embodiments, the composition or formulation has a viscosity of about 5000 cP to about 17,000 cP. In some embodiments, the composition or formulation has a viscosity of about 6000 cP to about 17,000 cP. In some embodiments, the composition or formulation has a viscosity of about 7000 cP to about 17,000 cP. In some embodiments, the composition or formulation has a viscosity of about 8000 cP to about 17,000 cP. In some embodiments, the composition or formulation has a viscosity of about 9000 cP to about 17,000 cP. In some embodiments, the composition or formulation has a viscosity of about 1000 cP to about 17,000 cP.

In some embodiments, the composition or formulation has a viscosity of about 100 cP to about 10,000 cP. In some embodiments, the composition or formulation has a viscosity of about 100 cP to about 5,000 cP. In some embodiments, the composition or formulation has a viscosity of about 500 cP to about 5,000 cP. In some embodiments, the composition or formulation has a viscosity of about 100, 200, 300, 400, 500, 600, 700, 800, 900, 1000, 1500, 2000, 2500, 3000, 3500, 4000, 4500, or 5000 cP, or more, including increments therein. In some embodiments, the composition or formulation is a pseudoplastic fluid (i.e., a fluid that can change viscosity depending on temperature, shear rate, and force).

In some embodiments, the composition or formulation further comprises one or more preservatives. Preservatives are used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Further, the pharmaceutical compositions disclosed herein may be incorporated into a skin patch for delivery of the drug directly onto the skin.

It will be appreciated that the actual dosages of the compounds disclosed herein, or pharmaceutically acceptable salts or esters thereof, will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

This amount will vary depending upon a variety of factors, including but not limited to the characteristics of the bioactive compositions and formulations disclosed herein (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier mg/kg or carriers in the formulation, and the route of administration. Further, an effective or therapeutically effective amount may vary depending on whether the one or more bioactive compositions and formulations disclosed herein is administered alone or in combination with other drug(s), other therapy/therapies or other therapeutic method(s) or modality/modalities. One skilled in the clinical and pharmacological arts will be able to determine an effective amount or therapeutically effective amount through routine experimentation, namely by monitoring a cell's or subject's response to administration of the one or more bioactive compositions and formulations disclosed herein and adjusting the dosage accordingly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of compounds disclosed herein, or a pharmaceutically acceptable salt or ester thereof, calculated to produce the desired therapeutic effect in association with the required pharmaceutical carrier. The specification for the dosage unit forms disclosed herein are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the presently disclosed methods.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. The embodiments disclosed herein are intended to encompass intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

### EXEMPLARY FORMULATIONS

TABLE 1

| | Formulation |
|---|---|
| Component | A (weight %) |
| Purified water | 82.35 |
| Glycerin | 2 |
| Disodium EDTA | 0.1 |
| 4-(((R)-1-carboxy-2- | 3 |
| (((2E,6E)-3,7,11- | |
| trimethyldodeca- | |
| 2,6,10-trien-1- | |
| yl)thio)ethyl)amino)- | |
| 4-oxobutanoic acid | |
| Propylene glycol | 5 |
| Transcutol P | 5 |
| Polysorbate 80 | 1 |
| Butylated | 0.1 |
| hydroxytoluene | |
| Methylparaben | 0.17 |
| Propylparaben | 0.03 |
| Hydroxyethylcellulose | 1.25 |
| Total | 100 |

TABLE 2

| | Formulation B | Formulation C | Formulation D |
|---|---|---|---|
| Component | (wt %) | (wt %) | (wt %) |
| 4-(((R)-1-carboxy-2- | 1.0 | 1.0 | 1.0 |
| (((2E,6E)-3,7,11- | | | |
| trimethyldodeca- | | | |
| 2,6,10-trien-1- | | | |
| yl)thio)ethyl)amino)- | | | |
| 4-oxobutanoic acid | | | |
| Propylene glycol, | 10.0 | 0.0 | 5.0 |
| USP | | | |
| Transcutol | 0.0 | 10.0 | 0.0 |
| Glycerin, USP | 2.0 | 2.0 | 2.0 |
| Methylparaben, NF | 0.17 | 0.17 | 0.17 |
| Propylparaben, NF | 0.03 | 0.03 | 0.03 |
| Benzyl alcohol NF | 0.0 | 0.0 | 0.0 |
| Disodium EDTA, | 0.1 | 0.1 | 0.1 |
| USP | | | |
| Sodium metabisufite | 0.0 | 0.0 | 0.0 |
| Butylated | 0.1 | 0.1 | 0.0 |
| hydroxyanisole, NF | | | |
| (BHA) | | | |
| tert- | 0.0 | 0.0 | 0.0 |
| Butylhydroquinone, | | | |
| FCC (TBHQ) | | | |
| Tween 80 | 1.0 | 1.0 | 0.0 |
| Carbopol 981 | 0.85 | 0.85 | 0.85 |
| Hydroxyethyl | 0.0 | 0.0 | 0.0 |
| cellulose HHX | | | |
| Purified water, USP | QSad | QSad | QSad |
| Phosphate buffer pH | 0.0 | 0.0 | 0.0 |
| 7.0 (25 mM) | | | |
| Tromethamine 25% | QSad pH 7 | QSad pH 7 | QSad pH 7 |
| solution | | | |
| 4% NaOH solution | 0.0 | 0.0 | 0.0 |
| Dilute HCl solution, | QSad pH 7 | QSad pH 7 | QSad pH 7 |
| NF | | | |
| Total | 100.0 | 100.0 | 100.0 |

TABLE 3

| | Formulation E | Formulation F | Formulation G |
|---|---|---|---|
| Component | (wt %) | (wt %) | (wt %) |
| 4-(((R)-1-carboxy-2- | 1.0 | 1.0 | 1.0 |
| (((2E,6E)-3,7,11- | | | |
| trimethyldodeca- | | | |
| 2,6,10-trien-1- | | | |
| yl)thio)ethyl)amino)- | | | |
| 4-oxobutanoic acid | | | |
| Propylene glycol, | 10.0 | 5.0 | 5.0 |
| USP | | | |
| Transcutol | 0.0 | 0.0 | 0.0 |
| Glycerin, USP | 2.0 | 2.0 | 2.0 |
| Methylparaben, NF | 0.17 | 0.17 | 0.17 |
| Propylparaben, NF | 0.03 | 0.03 | 0.03 |
| Benzyl alcohol NF | 0.0 | 0.0 | 0.0 |
| Disodium EDTA, | 0.1 | 0.1 | 0.1 |
| USP | | | |
| Sodium metabisufite | 0.0 | 0.0 | 0.0 |
| Butylated | 0.1 | 0.1 | 0.1 |
| hydroxyanisole, NF | | | |
| (BHA) | | | |
| tert- | 0.0 | 0.0 | 0.02 |
| Butylhydroquinone, | | | |
| FCC (TBHQ) | | | |
| Tween 80 | 0.5 | 1.0 | 0.0 |
| Carbopol 981 | 0.85 | 0.85 | 0.85 |
| Hydroxyethyl | 0.0 | 0.0 | 0.0 |
| cellulose HHX | | | |
| Purified water, USP | QSad | QSad | QSad |
| Phosphate buffer pH | 0.0 | 0.0 | 0.0 |
| 7.0 (25 mM) | | | |
| Tromethamine 25% | QSad pH 7 | QSad pH 7 | QSad pH 7 |
| solution | | | |
| 4% NaOH solution | 0.0 | 0.0 | 0.0 |
| Dilute HCl solution, | QSad pH 7 | QSad pH 7 | QSad pH 7 |
| NF | | | |
| Total | 100.0 | 100.0 | 100.0 |

TABLE 4

| | Formulation H | Formulation I | Formulation J |
|---|---|---|---|
| Component | (wt %) | (wt %) | (wt %) |
| 4-(((R)-1-carboxy-2- | 1.0 | 5.0 | 1.0 |
| (((2E,6E)-3,7,11- | | | |
| trimethyldodeca- | | | |
| 2,6,10-trien-1- | | | |
| yl)thio)ethyl)amino)- | | | |
| 4-oxobutanoic acid | | | |
| Propylene glycol, | 5.0 | 10.0 | 10.0 |
| USP | | | |
| Transcutol | 0.0 | 0.0 | 0.0 |
| Glycerin, USP | 2.0 | 2.0 | 2.0 |
| Methylparaben, NF | 0.17 | 0.17 | 0.0 |
| Propylparaben, NF | 0.03 | 0.03 | 0.0 |
| Benzyl alcohol NF | 0.0 | 0.0 | 1.0 |
| Disodium EDTA, | 0.1 | 0.1 | 0.1 |
| USP | | | |
| Sodium metabisufite | 0.0 | 0.0 | 0.0 |
| Butylated | 0.0 | 0.1 | 0.1 |
| hydroxyanisole, NF | | | |
| (BHA) | | | |
| tert- | 0.02 | 0.0 | 0.0 |
| Butylhydroquinone, | | | |
| FCC (TBHQ) | | | |
| Tween 80 | 1.0 | 1.0 | 1.0 |
| Carbopol 981 | 0.85 | 0.85 | 0.85 |
| Hydroxyethyl | 0.0 | 0.0 | 0.0 |
| cellulose HHX | | | |
| Purified water, USP | QSad | QSad | QSad |
| Phosphate buffer pH | 0.0 | 0.0 | 0.0 |
| 7.0 (25 mM) | | | |
| Tromethamine 25% | QSad pH 7 | QSad pH 7 | QSad pH 7 |
| solution | | | |
| 4% NaOH solution | 0.0 | 0.0 | 0.0 |
| Dilute HCl solution, | QSad pH 7 | QSad pH 7 | QSad pH 7 |
| NF | | | |
| Total | 100.0 | 100.0 | 100.0 |

TABLE 5

| | Formulation K | Formulation L | Formulation M |
|---|---|---|---|
| Component | (wt %) | (wt %) | (wt %) |
| 4-(((R)-1-carboxy-2- | 1.0 | 1.0 | 1.0 |
| (((2E,6E)-3,7,11- | | | |
| trimethyldodeca- | | | |
| 2,6,10-trien-1- | | | |
| yl)thio)ethyl)amino)- | | | |
| 4-oxobutanoic acid | | | |
| Propylene glycol, | 10.0 | 10.0 | 5.0 |
| USP | | | |
| Transcutol | 0.0 | 0.0 | 0.0 |
| Glycerin, USP | 2.0 | 2.0 | 2.0 |
| Methylparaben, NF | 0.0 | 0.17 | 0.17 |
| Propylparaben, NF | 0.0 | 0.03 | 0.03 |
| Benzyl alcohol NF | 1.0 | 0.0 | 0.0 |
| Disodium EDTA, | 0.1 | 0.1 | 0.1 |
| USP | | | |
| Sodium metabisufite | 0.0 | 0.0 | 0.0 |
| Butylated | 0.1 | 0.1 | 0.1 |
| hydroxyanisole, NF | | | |
| (BHA) | | | |
| tert- | 0.0 | 0.0 | 0.0 |
| Butylhydroquinone, | | | |
| FCC (TBHQ) | | | |
| Tween 80 | 1.0 | 1.0 | 1.0 |
| Carbopol 981 | 0.0 | 0.0 | 0.0 |
| Hydroxyethyl | 1.25 | 1.25 | 1.25 |
| cellulose HHX | | | |
| Purified water, USP | QSad | QSad | QSad |
| Phosphate buffer pH | QSad pH 7 | 0.0 | 0.0 |
| 7.0 (25 mM) | | | |
| Tromethamine 25% | 0.0 | 0.0 | 0.0 |
| solution | | | |
| 4% NaOH solution | QSad pH 7 | QSad pH 7 | QSad pH 7 |
| Dilute HCl solution, | QSad pH 7 | QSad pH 7 | QSad pH 7 |
| NF | | | |
| Total | 100.0 | 100.0 | 100.0 |

TABLE 6

| | Formulation N | Formulation O | Formulation P |
|---|---|---|---|
| Component | (wt %) | (wt %) | (wt %) |
| 4-(((R)-1-carboxy-2- | 1.0 | 1.0 | 1.0 |
| (((2E,6E)-3,7,11- | | | |
| trimethyldodeca- | | | |
| 2,6,10-trien-1- | | | |
| yl)thio)ethyl)amino)- | | | |
| 4-oxobutanoic acid | | | |
| Propylene glycol, | 5.0 | 5.0 | 5.0 |
| USP | | | |
| Transcutol | 0.0 | 0.0 | 5.0 |
| Glycerin, USP | 2.0 | 2.0 | 2.0 |
| Methylparaben, NF | 0.17 | 0.17 | 0.17 |
| Propylparaben, NF | 0.03 | 0.03 | 0.03 |
| Benzyl alcohol NF | 0.0 | 0.0 | 0.0 |
| Disodium EDTA, | 0.1 | 0.1 | 0.1 |
| USP | | | |
| Sodium metabisufite | 0.0 | 0.0 | 0.0 |
| Butylated | 0.05 | 0.01 | 0.1 |
| hydroxyanisole, NF | | | |
| (BHA) | | | |
| tert- | 0.0 | 0.0 | 0.0 |
| Butylhydroquinone, | | | |
| FCC (TBHQ) | | | |
| Tween 80 | 1.0 | 1.0 | 1.0 |
| Carbopol 981 | 0.0 | 0.0 | 0.0 |
| Hydroxyethyl | 1.25 | 1.25 | 1.25 |
| cellulose HHX | | | |
| Purified water, USP | QSad | QSad | QSad |
| Phosphate buffer pH | 0.0 | 0.0 | 0.0 |
| 7.0 (25 mM) | | | |
| Tromethamine 25% | 0.0 | 0.0 | 0.0 |
| solution | | | |
| 4% NaOH solution | QSad pH 7 | QSad pH 7 | QSad pH 7 |
| Dilute HCl solution, | QSad pH 7 | QSad pH 7 | QSad pH 7 |
| NF | | | |
| Total | 100.0 | 100.0 | 100.0 |

TABLE 7

| | Formulation Q | Formulation R |
|---|---|---|
| Component | (wt %) | (wt %) |
| 4-(((R)-1-carboxy-2- | 1.0 | 1.0 |
| (((2E,6E)-3,7,11- | | |
| trimethyldodeca- | | |
| 2,6,10-trien-1- | | |
| yl)thio)ethyl)amino)- | | |
| 4-oxobutanoic acid | | |
| Propylene glycol, | 0.0 | 10.0 |
| USP | | |
| Transcutol | 10.0 | 0.0 |
| Glycerin, USP | 2.0 | 2.0 |
| Methylparaben, NF | 0.17 | 0.17 |
| Propylparaben, NF | 0.03 | 0.03 |
| Benzyl alcohol NF | 0.0 | 0.0 |
| Disodium EDTA, | 0.1 | 0.1 |
| USP | | |
| Sodium metabisufite | 0.0 | 0.0 |
| Butylated | 0.1 | 0.1 |
| hydroxyanisole, NF | | |
| (BHA) | | |
| tert- | 0.0 | 0.0 |
| Butylhydroquinone, | | |
| FCC (TBHQ) | | |
| Tween 80 | 1.0 | 1.0 |
| Carbopol 981 | 0.0 | 0.0 |
| Hydroxyethyl | 1.25 | 0.6 |
| cellulose HHX | | |
| Purified water, USP | QSad | QSad |
| Phosphate buffer pH | 0.0 | 0.0 |
| 7.0 (25 mM) | | |
| Tromethamine 25% | 0.0 | 0.0 |
| solution | | |
| 4% NaOH solution | QSad pH 7 | QSad pH 7 |
| Dilute HCl solution, | QSad pH 7 | QSad pH 7 |
| NF | | |
| Total | 100.0 | 100.0 |

TABLE 8

| | Formu- | Formu- | Formu- | Formu- |
|---|---|---|---|---|
| | lation S | lation T | lation U | lation V |
| Component | (wt %) | (wt %) | (wt %) | (wt %) |
| Emulsifying wax, | 12.0 | | | |
| NF | | | | |
| Brij 721 (PEG 21 | 0.0 | 2.0 | 4.0 | 0.0 |
| stearyl ether) | | | | |
| Brij 72 (PEG 2 | 0.0 | 2.0 | 1.0 | 0.0 |
| stearyl ether) | | | | |
| Pemulen TR1 | 0.0 | 0.0 | 0.0 | 0.3 |
| White petrolatum, | 5.0 | 10 | 0.0 | 0.0 |
| NF | | | | |
| Myristyl lactate, NF | 5.0 | 0.0 | 8.8 | 7.2 |
| Diisopropyl adipate | 0.0 | 5.0 | 0.0 | 5.0 |
| Cetyl Alcohol, NF | 0.0 | 7.0 | 7.0 | 0.0 |
| Cyclomethicone, NF | 4.8 | 4.8 | 2.0 | 10.0 |
| Oleyl alcohol, NF | 2.0 | 2.0 | 2.0 | 2.0 |
| Cholesterol, NF | 1.0 | 0.0 | 0.0 | 0.0 |
| Brij 30 (Laureth-4, | 0.0 | 0.0 | 0.0 | 0.3 |
| POE lauryl ether) | | | | |
| Butylated | 0.1 | 0.1 | 0.1 | 0.1 |
| hydroxytoluene, NF | | | | |
| (BHT) | | | | |
| Butylated | 0.1 | 0.1 | 0.1 | 0.1 |
| hydroxyanisole, NF | | | | |
| (BHA) | | | | |
| Disodium 4-(((R)-1- | 1.0 | 1.0 | 1.0 | 1.0 |
| carboxylato-2- | | | | |
| (((2E,6E)-3,7,11- | | | | |
| trimethyldodeca- | | | | |
| 2,6,10-trien-1- | | | | |
| yl)thio)ethyl)amino)- | | | | |
| 4-oxobutanoate | | | | |
| Propylene glycol, | 10.0 | 5.0 | 10.0 | 10.0 |
| USP | | | | |
| Methylparaben, NF | 0.17 | 0.17 | 0.17 | 0.17 |
| Propylparaben, NF | 0.03 | 0.03 | 0.03 | 0.03 |
| EDTA, USP | 0.1 | 0.1 | 0.1 | 0.1 |
| Purified water | QSad | QSad | QSad | QSad |
| Carbopol 980 | 0.4 | 0.4 | 0.4 | 0.6 |
| 4% NaOH Solution | QSad | QSad | QSad | |
| | pH 7 | pH 7 | pH 7 | |
| Dilute HCl solution, | QSad | QSad | QSad | QSad |
| NF | pH 7 | pH 7 | pH 7 | pH 7 |
| 10% NaOH Solution | | | | QSad |
| | | | | pH 7 |
| Total | 100.00 | 100.00 | 100.00 | 100.00 |

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc, are presented merely for ease of reading the specification and claims. The use of headings in the specification or claims does not require the steps or elements be performed in alphabetical or numerical order or the order in which they are presented.

The preparations and examples of a number of embodiments are intended to be illustrative and not limiting.

The preparation of compounds 4-((1-carboxy-2-(((2E,6E)-3,7,11-trimethyldodeca-2,6,10-trien-1-yl)thio)ethyl)amino)-4-oxobutanoic acid, 4-(((R)-1-carboxy-2-(((2E,6E)-3,7,11-trimethyldodeca-2,6,10-trien-1-yl)thio)ethyl)amino)-4-oxobutanoic acid, and 4-(((S)-1-carboxy-2-(((2E,6E)-3,7,11-trimethyldodeca-2,6,10-trien-1-yl)thio)ethyl)amino)-4-oxobutanoic acid, and pharmaceutically acceptable salts thereof, are disclosed in U.S. Pat. Nos. 8,372,884 and 8,461,204, the contents of which are hereby incorporated by reference in their entirety.

Other materials used in the preparation of the pharmaceutical compositions disclosed herein are available commercially or are described in the literature. All temperatures are reported in ° C.

### Example 1

Formulations B-V, set forth in Tables 2-8, were prepared and subjected to a four-week physical and chemical stability analysis. Appearance and pH testing was performed on all formulations on stability. Thirteen formulations including 10 gels and 3 creams were submitted for chemical assay. Formulation selection for HPLC analysis was based on initial data, physical observations at four weeks and compositional variables.

Physical evaluation of samples stored under freeze/thaw (FT) and 5° C. was conducted to assess whether physical instability (e.g. precipitation and/or phase separation) occurs under typical shipping conditions. F/T also provides a significant ‘accelerated’ stress condition. The 5° C. condition provides an indication whether formulation components such as API's or antioxidants (e.g. BHA) were formulated at concentrations closer to saturation than optimal.

As for chemical assay methodology, a single sample preparation of each formulation was prepared in the 4 weeks sampling interval as supported by duplicate data generated at the initial time point.

Results are shown below in Tables 9-13. Data indicated that a large proportion of the gel and cream compositions were more stable at 25° C. relative to the 40° C. condition. pH remained consistent in the formulae irrespective of formulation type or storage condition. Syneresis/phase separation was not observed in any of the formulae. Significant orange/pink color formation was observed in compositions containing TBHQ. However, little or no color change was observed in other compositions. A slight color change was noted in Formulation J and this may be associated with the presence of Benzyl alcohol. However, such a slight color change may not be discernable to a patient when extruding the formulation from a tube.

With the exception of the color changes noted with TBHQ formulations (Formulations G and H), the formulations exhibited favorable results. Formulations Q and T exhibited particularly favorable chemical stability at 25° C. and 40° C., with essentially no change from initial. Formulations O, J and V also demonstrated particularly favorable physical stability at 25° C. and 40° C., with little to no change in the initial appearance of a white cream. With the exception Formulation U and Formulation V, the sulfoxide content remained close to 1% LC or less. The positive chemical stability correlated well with in vitro skin permeation data where Formulation J and Formulation T produced high receptor and tissue concentrations.

TABLE 9

| | | | | | | | Assay % LC | | |
|---|---|---|---|---|---|---|---|---|---|
| | | | | | | | 4-(((R)-1-carboxy-2- | | |
| | | | | | | | (((2E,6E)-3,7,11- | | |
| | | | | | | | trimethyldodeca- | | |
| | | | | | | | 2,6,10-trien-1- | Primary | |
| Formulation | Formulation | | | Physical | | | yl)thio)ethyl)amino)- | sulfoxide | Total |
| type | ID | Interval | Condition | Observation | pH (neat) | Viscosity | 4-oxobutanoic acid | deg* | degs* |
| Carbopol | B | Initial | CRT | clear light | 6.61 | 6660 | 99.9 | 0.3 | 2.4 |
| gel | | | | yellow gel | | | | | |
| | | 4 | F/T | NC | 6.66 | N/T | N/T | | |
| | | weeks | 5° C. | NC | 6.59 | | | | |
| | | | 25° C. | NC | 6.61 | | 99.2 | 0.5 | 2.7 |
| | | | 40° C. | NC | 6.66 | | 87.7 | 0.7 | 13.6 |
| | C | Initial | CRT | clear light | 6.61 | 5860 | 100.6 | 0.4 | 2.7 |
| | | | | yellow gel | | | | | |
| | | 4 | F/T | NC | 6.68 | N/T | N/T | | |
| | | weeks | 5° C. | NC | 6.62 | | | | |
| | | | 25° C. | NC | 6.71 | | 94.9 | 0.5 | 4.0 |
| | | | 40° C. | NC | 6.70 | | 88.0 | 0.7 | 12.6 |
| | D | Initial | CRT | clear | 6.64 | 6250 | 97.9 | 0.5 | 1.1 |
| | | | | colorless | | | | | |
| | | | | gel | | | | | |
| | | 4 | F/T | NC | 6.77 | N/T | N/T | | |
| | | weeks | 5° C. | NC | 6.70 | | | | |
| | | | 25° C. | NC | 6.74 | | 96.5 | NRP | 3.5 |
| | | | 40° C. | NC | 6.79 | | 90.4 | 0.2 | 10.2 |
| | E | Initial | CRT | clear light | 6.81 | 6400 | 98.9 | 0.3 | 2.2 |
| | | | | yellow gel | | | | | |
| | | 4 | F/T | NC | 6.82 | N/T | N/T | | |
| | | weeks | 5° C. | NC | 6.76 | | | | |
| | | | 25° C. | NC | 6.83 | | | | |
| | | | 40° C. | hazy white | 6.85 | | | | |
| | | | | gel | | | | | |
| | F | Initial | CRT | clear light | 6.73 | 7120 | 98.8 | 0.5 | 2.9 |
| | | | | yellow gel | | | | | |

| | | | | | |
|---|---|---|---|---|---|
| 4 | F/T | NC | 6.72 | N/T | N/T |
| weeks | 5° C. | NC | 6.67 | | |

| | | | | | |
|---|---|---|---|---|---|
| 25° C. | NC | 6.70 | 98.6 | 0.3 | 4.4 |
| 40° C. | NC | 6.73 | 87.0 | 0.6 | 13.1 |

TABLE 10

| | | | | | | | Assay % LC | | |
|---|---|---|---|---|---|---|---|---|---|
| | | | | | | | 4-(((R)-1- | | |
| | | | | | | | carboxy-2- | | |
| | | | | | | | (((2E,6E)-3,7,11- | | |
| | | | | | | | trimethyldodeca- | | |
| | | | | | | | 2,6,10-trien-1- | | |
| | | | | | | | yl)thio)ethyl)amino)- | | |
| Formulation | Formulation | | | Physical | | | 4- | Primary | Total |
| type | ID | Interval | Condition | Observation | pH (neat) | Viscosity | oxobutanoic acid | sulfoxide deg* | degs* |
| Carbopol | G | Initial | CRT | clear | 6.88 | 7210 | 98.2 | 0.3 | 1.0 |
| gel | | | | colorless | | | | | |
| (TBHQ) | | | | gel | | | | | |
| | | 4 weeks | F/T | clear pink- | 6.78 | N/T | N/T | | |
| | | | | orange gel | | | | | |
| | | | 5° C. | clear | 6.75 | | | | |
| | | | | slightly | | | | | |
| | | | | pink gel | | | | | |
| | | | 25° C. | clear pink- | 6.80 | | | | |
| | | | | orange gel | | | | | |
| | | | 40° C. | slightly | 6.73 | | | | |
| | | | | hazy orange | | | | | |
| | | | | gel | | | | | |
| | H | Initial | CRT | clear light | 6.86 | 5650 | 100.3 | 0.3 | 1.4 |
| | | | | yellow gel | | | | | |
| | | 4 weeks | F/T | clear pink- | 6.81 | N/T | N/T | | |
| | | | | orange gel | | | | | |
| | | | 5° C. | clear | 6.82 | | | | |
| | | | | slightly | | | | | |
| | | | | pink gel | | | | | |
| | | | 25° C. | clear pink- | 6.83 | | | | |
| | | | | orange gel | | | | | |
| | | | 40° C. | clear | 6.74 | | | | |
| | | | | orange gel | | | | | |
| Carbopol | I | Initial | CRT | hazy light | 7.05 | 1550 | 103.8 | 0.2 | 1.9 |
| gel | | | | yellow gel | | | | | |
| (5% | | 4 weeks | F/T | NC | 7.02 | N/T | N/T | | |
| API) | | | 5° C. | NC | 7.07 | | | | |
| | | | 25° C. | NC | 7.08 | | | | |
| | | | 40° C. | hazy yellow | 7.09 | | | | |
| | | | | gel | | | | | |
| Carbopol | J | Initial | CRT | clear light | 7.47 | 6470 | 102.3 | 0.4 | 2.6 |
| gel | | | | yellow gel | | | | | |
| | | 4 weeks | F/T | NC | 7.35 | N/T | N/T | | |
| | | | 5° C. | NC | 7.30 | | | | |
| | | | 25° C. | NC | 7.34 | | 101.0 | 0.3 | 3.2 |
| | | | 40° C. | clear | 7.32 | | 96.1 | 1.0 | 9.0 |
| | | | | slightly | | | | | |
| | | | | pink gel | | | | | |
| HEC gel | K | Initial | CRT | clear light | 7.06 | 15600 | 100.2 | 0.3 | 3.8 |
| (Phosphate | | | | yellow gel | | | | | |

| | | | | | | |
|---|---|---|---|---|---|---|
| buffer) | 4 weeks | F/T | NC | 7.10 | N/T | N/T |
| | | 5° C. | NC | 7.07 | | |
| | | 25° C. | NC | 7.08 | | |
| | | 40° C. | clear | 7.10 | | |
| | | | slightly | | | |
| | | | pink gel | | | |

TABLE 11

| | | | | | | | Assay % LC | | |
|---|---|---|---|---|---|---|---|---|---|
| | | | | | | | 4-(((R)-1- | | |
| | | | | | | | carboxy-2- | | |
| | | | | | | | (((2E,6E)-3,7,11- | | |
| | | | | | | | trimethyldodeca- | | |
| | | | | | | | 2,6,10-trien-1- | | |
| | | | | | | | yl)thio)ethyl)amino)- | | |
| Formulation | Formulation | | | Physical | | | 4- | Primary | Total |
| type | ID | Interval | Condition | Observation | pH (neat) | Viscosity | oxobutanoic acid | sulfoxide deg* | degs* |
| HEC gel | L | Initial | CRT | clear light | 6.80 | 15700 | 99.5 | 0.4 | 3.9 |
| | | | | yellow gel | | | | | |
| | | 4 weeks | F/T | NC | 7.00 | N/T | N/T | | |
| | | | 5° C. | NC | 6.93 | | | | |
| | | | 25° C. | NC | 6.92 | | 99.1 | 0.5 | 3.6 |
| | | | 40° C. | NC | 6.96 | | 92.1 | 1.1 | 12.1 |
| | M | Initial | CRT | slightly | 6.82 | 13340 | 99.4 | 0.2 | 3.1 |
| | | | | hazy and | | | | | |
| | | | | light yellow | | | | | |
| | | | | gel | | | | | |
| | | 4 weeks | F/T | NC | 6.91 | N/T | N/T | | |
| | | | 5° C. | NC | 6.90 | | | | |
| | | | 25° C. | NC | 6.95 | | 99.0 | 0.5 | 4.4 |
| | | | 40° C. | NC | 6.96 | | 90.8 | 0.9 | 12.1 |
| | N | Initial | CRT | clear light | 6.79 | 14400 | 100.7 | 0.4 | 4.0 |
| | | | | yellow gel | | | | | |
| | | 4 weeks | F/T | NC | 6.94 | N/T | N/T | | |
| | | | 5° C. | NC | 6.89 | | | | |
| | | | 25° C. | NC | 6.97 | | 101.2 | 0.4 | 3.5 |
| | | | 40° C. | NC | 6.98 | | 91.5 | 0.8 | 11.6 |
| | O | Initial | CRT | clear light | 6.80 | 14600 | 101.8 | 0.4 | 2.9 |
| | | | | yellow gel | | | | | |
| | | 4 weeks | F/T | NC | 6.92 | N/T | N/T | | |
| | | | 5° C. | NC | 6.92 | | | | |
| | | | 25° C. | NC | 6.98 | | 99.7 | 0.4 | 4.2 |
| | | | 40° C. | NC | 6.98 | | 93.8 | 0.6 | 11.5 |
| | P | Initial | CRT | clear light | 6.81 | 16020 | 99.4 | 0.3 | 3.4 |
| | | | | yellow gel | | | | | |

| | | | | | |
|---|---|---|---|---|---|
| 4 weeks | F/T | NC | 6.93 | N/T | N/T |
| | 5° C. | NC | 6.88 | | |
| | 25° C. | NC | 6.94 | | |
| | 40° C. | NC | 6.99 | | |

TABLE 12

| | | | | | | | Assay % LC | | |
|---|---|---|---|---|---|---|---|---|---|
| | | | | | | | 4-(((R)-1-carboxy- | | |
| | | | | | | | 2-(((2E,6E)-3,7,11- | | |
| | | | | | | | trimethyldodeca- | | |
| | | | | | | | 2,6,10-trien-1- | | |
| | | | | | | | yl)thio)ethyl)amino)- | Primary | |
| Formulation | Formulation | | | Physical | | | 4-oxobutanoic | sulfoxide | Total |
| type | ID | Interval | Condition | Observation | pH (neat) | Viscosity | acid | deg* | degs* |
| HEC gel | Q | Initial | CRT | clear light | 6.85 | 14500 | 99.9 | 0.5 | 4.0 |
| | | | | yellow gel | | | | | |
| | | 4 weeks | F/T | NC | 6.91 | N/T | N/T | | |
| | | | 5° C. | NC | 6.87 | | | | |
| | | | 25° C. | NC | 6.93 | | 100.8 | 0.6 | 4.4 |
| | | | 40° C. | NC | 6.99 | | 97.1 | 1.2 | 12.2 |
| | R | Initial | CRT | clear light | 6.87 | 1360 | 100.8 | 0.4 | 4.9 |
| | | | | yellow gel | | | | | |
| | | 4 weeks | F/T | NC | 6.96 | N/T | N/T | | |
| | | | 5° C. | NC | 6.89 | | | | |
| | | | 25° C. | NC | 6.92 | | | | |
| | | | 40° C. | NC | 6.99 | | | | |
| Emulsifying | S | Initial | CRT | white | 6.66 | 37700 | 82.3 | 19.3 | 57.5 |
| wax | | | | cream | | | | | |
| cream | | 4 weeks | F/T | NC | 6.61 | N/T | N/T | | |
| | | | 5° C. | NC | 6.60 | | | | |
| | | | 25° C. | NC | 6.56 | | | | |
| | | | 40° C. | slightly | 6.43 | | | | |
| | | | | yellow | | | | | |
| | | | | cream | | | | | |
| Brij 1:1 | T | Initial | CRT | white | 6.70 | 4970 | 98 | 1.1 | 40.5 |
| cream | | | | cream | | | | | |

| | | | | | |
|---|---|---|---|---|---|
| 4 weeks | F/T | NC | 6.81 | N/T | N/T |
| | 5° C. | NC | 6.80 | | |

| | | | | | |
|---|---|---|---|---|---|
| 25° C. | NC | 6.84 | 100.4 | 1.3 | 43.6 |
| 40° C. | NC | 6.82 | 98.4 | 1.1 | 56.6 |

TABLE 13

| | | | | | | | Assay % LC | | |
|---|---|---|---|---|---|---|---|---|---|
| | | | | | | | 4-(((R)-1-carboxy- | | |
| | | | | | | | 2-(((2E,6E)-3,7,11- | | |
| | | | | | | | trimethyldodeca- | | |
| | | | | | | | 2,6,10-trien-1- | | |
| | | | | | | | yl)thio)ethyl)amino)- | Primary | |
| Formulation | Formulation | | | Physical | pH | | 4-oxobutanoic | sulfoxide | |
| type | ID | Interval | Condition | Observation | (neat) | Viscosity | acid | deg* | Total degs* |
| Brij 4:1 | U | Initial | CRT | white | 6.82 | 14600 | 96.6 | 5.6 | 36.9 |
| cream | | | | cream | | | | | |
| | | 4 | F/T | NC | 6.71 | N/T | N/T | | |
| | | weeks | 5° C. | NC | 6.75 | | | | |
| | | | 25° C. | NC | 6.64 | | 94.2 | 5.4 | 43.2 |
| | | | 40° C. | NC | 6.46 | | 76.2 | 4.4 | 55.6 |
| Pemulen | V | Initial | CRT | white | 6.96 | 36350 | 102.5 | 4.7 | 46.6 |
| cream | | | | cream | | | | | |

| | | | | | |
|---|---|---|---|---|---|
| 4 | F/T | NC | 7.04 | N/T | N/T |
| weeks | 5° C. | NC | 7.04 | | |

| | | | | | |
|---|---|---|---|---|---|
| 25° C. | NC | 7.03 | 103.1 | 4.4 | 51.4 |
| 40° C. | off-white | 6.93 | 94.7 | 5.5 | 54.2 |
| | cream | | | | |

Legend - Tables 9-13
% LC—% Label claim
*Assumes identical response factor to Sig990. Deg(s)—Degradation product.
Total degs - sum of the sulfoxide and unknown degradants individually above 0.5% LC
NRP—not reportable peaks
NC - no change relative to initial
N/T—not tested

### Example 2

The stability of a formulation comprising the disodium salt of 4-(((R)-1-carboxy-2-(((2E,6E)-3,7,11-trimethyldodeca-2,6,10-trien-1-yl)thio)ethyl)amino)-4-oxobutanoic acid was determined after storage at 25° C. for a period of 4 months, 6 months, 9 months and 12 months. In each case, the percentage of 4-(((R)-1-carboxy-2-(((2E,6E)-3,7,11-trimethyldodeca-2,6,10-trien-1-yl)thio)ethyl)amino)-4-oxobutanoic acid remaining at each time point was determined by use of HPLC. The results are presented in Table 14.

TABLE 14

| | Initial | % weight in | % weight in | % weight in | % weight in |
|---|---|---|---|---|---|
| | (% weight of | formulation | formulation | formulation | formulation |
| Component | formulation) | after 1 month | after 3 months | after 6 months | after 9 months |
| Methylparaben | 0.17% | 0.17% | 0.17% | 0.17% | 0.16% |
| Propylparaben | 0.034% | 0.033% | 0.034% | 0.032% | 0.033% |
| 4-(((R)-1-carboxy-2-(((2E,6E)- | 3.09% | 2.97% | 3.13% | 2.92% | 2.87% |
| 3,7,11-trimethyldodeca-2,6,10- | | | | | |
| trien-1-yl)thio)ethyl)amino)- | | | | | |
| 4-oxobutanoic acid | | | | | |

% age of sulfoxide of 4-(((R)-1-carboxy-2-(((2E,6E)-3,7,11-
trimethyldodeca-2,6,10-trien-1-yl)thio)ethyl)amino)-4-oxobutanoic acid

| | | | | |
|---|---|---|---|---|
| 0.037% | 0.031% | 0.113% | 0.053% | 0.134% |

### Example 3

The stability of a formulation comprising the disodium salt of 4-(((R)-1-carboxy-2-(((2E,6E)-3,7,11-trimethyldodeca-2,6,10-trien-1-yl)thio)ethyl)amino)-4-oxobutanoic acid was determined after storage at 5° C. for a period of 1 month, 2 months, 3 months, 6 months, 9 months, 12 months, 18 months, and 24 months. In each case, the percentage of 4-(((R)-1-carboxy-2-(((2E,6E)-3,7,11-trimethyldodeca-2,6,10-trien-1-yl)thio)ethyl)amino)-4-oxobutanoic acid remaining at each time point was determined by use of HPLC. Each of the percentages in the table below are expressed at the weight percentage of the component as compared to the total weight of the composition. The results are presented in Table 15.

TABLE 15

| | months | | | | | | | | |
|---|---|---|---|---|---|---|---|---|---|
| Component | Initial | 1 | 2 | 3 | 6 | 9 | 12 | 18 | 24 |
| Methylparaben | 0.17 | 0.17 | 0.17 | 0.17 | 0.18 | 0.17 | 0.17 | 0.17 | 0.15 |
| Propylparaben | 0.034 | 0.032 | 0.032 | 0.033 | 0.032 | 0.033 | 0.032 | 0.033 | 0.030 |

1. A pharmaceutical composition comprising (a) at least one protective agent selected from the group consisting of butylated hydroxyanisole, butylated hydroxytoluene, sodium metabisulfite, tert-butylhydroquinone, methylparaben, propylparaben, benzyl alcohol, poly(acrylic acid), hydroxyethyl cellulose, emulsifying wax, PEG-21 stearyl ether, PEG-2 stearyl ether, white petrolatum, myristyl lactate, diisopropyl adipate, cetyl alcohol, cyclomethicone, oleyl alcohol, cholesterol, and polyoxyethylene(4)lauryl ether; and (b) a therapeutically effective amount of an IPC Active Agent or a pharmaceutically acceptable salt or ester thereof.

2. The pharmaceutical composition of claim 1, wherein the protective agent is selected from the group consisting of butylated hydroxyanisole, butylated hydroxytoluene, sodium metabisulfite, tert-butylhydroquinone, methylparaben, propylparaben, and poly(acrylic acid).

3. The pharmaceutical composition of claim 1, wherein the protective agent includes butylated hydroxyanisole.

4. The pharmaceutical composition of claim 3, wherein the butylated hydroxyanisole is present in an amount from about 0.001% to about 2%, based on the total weight of the composition.

5. The pharmaceutical composition of claim 4, wherein the butylated hydroxyanisole is present in an amount from about 0.005% to about 1%, based on the total weight of the composition.

6. The pharmaceutical composition of claim 1, wherein the protective agent includes sodium metabisulfite.

7. The pharmaceutical composition of claim 6, wherein the sodium metabisulfite is present in an amount from about 0.01% to about 5%, based on the total weight of the composition.

8. The pharmaceutical composition of claim 7, wherein the butylated hydroxyanisole is present in an amount from about 0.05% to about 1%, based on the total weight of the composition.

9. The pharmaceutical composition of claim 1, wherein the protective agent includes tert-butylhydroquinone.

10. The pharmaceutical composition of claim 9, wherein the tert-butylhydroquinone is present in an amount from about 0.001% to about 2%, based on the total weight of the composition.

11. The pharmaceutical composition of claim 10, wherein the tert-butylhydroquinone is present in an amount from about 0.005% to about 1%, based on the total weight of the composition.

12. The pharmaceutical composition of claim 1, wherein the IPC Active Agent includes a compound depicted by Formula I: 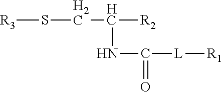
wherein:
L is a bivalent, branched or unbranched, saturated or unsaturated, C₂-C₆ hydrocarbon chain wherein one or more methylene units of L is independently replaced by —O—, —S—, —NH—, —C(O)—, —C═CH₂—, or C₃-C₆ cycloalkylene, wherein L is optionally substituted by one or more groups selected from halogen, phenyl, an 8-10 membered bicyclic aryl ring, a 5-6 membered heteroaryl ring having 1-4 heteroatoms independently selected from nitrogen, oxygen, or sulfur, an 8-10 membered bicyclic heteroaryl ring having 1-4 heteroatoms independently selected from nitrogen, oxygen, or sulfur, a 5- to 7-membered monocyclic having 1-2 heteroatoms independently selected from nitrogen, oxygen, or sulfur or a 7-10 membered bicyclic heterocyclyl ring having 1-2 heteroatoms independently selected from nitrogen, oxygen, or sulfur;
R₁ is hydrogen, —OH or —OR, wherein each R is independently hydrogen or an optionally substituted group selected from C₁₋₆ aliphatic or C₁₋₆ heteroaliphatic;
R₂ is —C(O)X, wherein X is independently R, —OR, a hydrogen, aryloxy, amino, alkylamino, dialkylamino, heteroaryloxy, hydrazine, a 6-10 membered aryl ring, a 5-6 membered heteroaryl ring having 1-4 heteroatoms independently selected from nitrogen, oxygen, or sulfur, wherein each R is independently hydrogen or an optionally substituted group selected from C₁₋₆ aliphatic or C₁₋₆ heteroaliphatic; and
R₃ is a substituted or unsubstituted, branched or unbranched, saturated or unsaturated, C₁₀-C₂₅ aliphatic,
or a pharmaceutically acceptable salt or ester thereof.

13. The pharmaceutical composition of claim 1, wherein the IPC Active Agent includes 4-((1-carboxy-2-(((2E,6E)-3,7,11-trimethyldodeca-2,6,10-trien-1-yl)thio)ethyl)amino)-4-oxobutanoic acid or a pharmaceutically acceptable salt or ester thereof.

14. The pharmaceutical composition of claim 1, wherein the IPC Active Agent includes the disodium salt of 4-((1-carboxy-2-(((2E,6E)-3,7,11-trimethyldodeca-2,6,10-trien-1-yl)thio)ethyl)amino)-4-oxobutanoic acid.

15. The pharmaceutical composition of claim 1, wherein the IPC Active Agent includes 4-(((R)-1-carboxy-2-(((2E,6E)-3,7,11-trimethyldodeca-2,6,10-trien-1-yl)thio)ethyl)amino)-4-oxobutanoic acid, or a pharmaceutically acceptable salt or ester thereof.

16. The pharmaceutical composition of claim 1, wherein the IPC Active Agent includes the disodium salt of 4-(((R)-1-carboxy-2-(((2E,6E)-3,7,11-trimethyldodeca-2,6,10-trien-1-yl)thio)ethyl)amino)-4-oxobutanoic acid.

17. The pharmaceutical composition of claim 1, wherein the IPC Active Agent includes 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt thereof.

18. The pharmaceutical composition of claim 1, wherein the IPC Active Agent includes the disodium salt of 4-(((S)-1-carboxy-2-(((2E,6E)-3,7,11-trimethyldodeca-2,6,10-trien-1-yl)thio)ethyl)amino)-4-oxobutanoic acid, or a pharmaceutically acceptable salt thereof.

19. The pharmaceutical composition of claim 1, wherein the IPC Active Agent includes at least 75 wt % of 4-(((R)-1-carboxy-2-(((2E,6E)-3,7,11-trimethyldodeca-2,6,10-trien-1-yl)thio)ethyl)amino)-4-oxobutanoic acid, or pharmaceutically acceptable salt or ester thereof, based on the total weight of 4-((1-carboxy-2-(((2E,6E)-3,7,11-trimethyldodeca-2,6,10-trien-1-yl)-thio)ethyl)amino)-4-oxobutanoic acid or a pharmaceutically acceptable salt or ester thereof present in the composition.

20-21. (canceled)

22. The pharmaceutical composition of claim 1, wherein the IPC Active Agent includes at least 90 wt % of 4-(((R)-1-carboxy-2-(((2E,6E)-3,7,11-trimethyldodeca-2,6,10-trien-1-yl)thio)ethyl)amino)-4-oxobutanoic acid, or pharmaceutically acceptable salt or ester thereof, based on the total weight of 4-((1-carboxy-2-(((2E,6E)-3,7,11-trimethyldodeca-2,6,10-trien-1-yl)-thio)ethyl)amino)-4-oxobutanoic acid or a pharmaceutically acceptable salt or ester thereof present in the composition.

23. (canceled)

24. The pharmaceutical composition of claim 1, wherein the IPC Active Agent includes at least 97.5 wt % of 4-(((R)-1-carboxy-2-(((2E,6E)-3,7,11-trimethyldodeca-2,6,10-trien-1-yl)thio)ethyl)amino)-4-oxobutanoic acid, or pharmaceutically acceptable salt or ester thereof, based on the total weight of 4-((1-carboxy-2-(((2E,6E)-3,7,11-trimethyldodeca-2,6,10-trien-1-yl)-thio)ethyl)amino)-4-oxobutanoic acid or a pharmaceutically acceptable salt or ester thereof present in the composition.

25. The pharmaceutical composition of claim 1, wherein the IPC Active Agent includes: 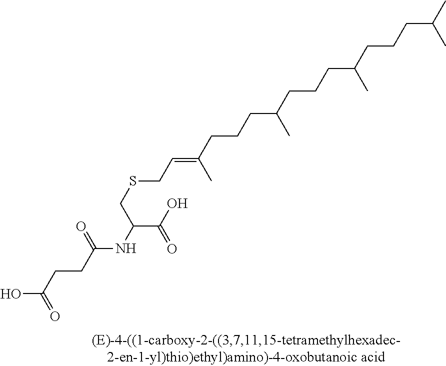 [SMILES: C/C(=C\CSCC(NC(=O)CCC(=O)O)C(=O)O)CCCC(C)CCCC(C)CCCC(C)C]
or a pharmaceutically acceptable salt or ester thereof.

26. The pharmaceutical composition of claim 1, wherein the IPC Active Agent includes at least 75 wt % of: 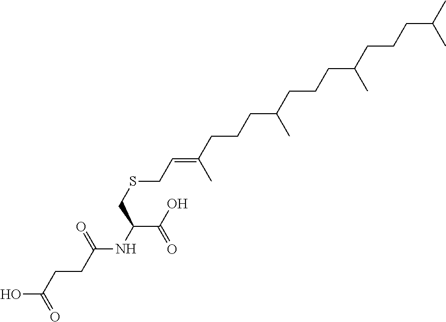 [SMILES: C/C(=C\CSC[C@H](NC(=O)CCC(=O)O)C(=O)O)CCCC(C)CCCC(C)CCCC(C)C]
or pharmaceutically acceptable salt or ester thereof, based on the total weight of (E)-4-((1-carboxy-2-((3,7,11,15-tetramethylhexadec-2-en-1-yl)thio)ethyl)amino)-4-oxobutanoic acid or a pharmaceutically acceptable salt or ester thereof present in the composition.

27-28. (canceled)

29. The pharmaceutical composition of claim 1, wherein the IPC Active Agent includes at least 90 wt % of 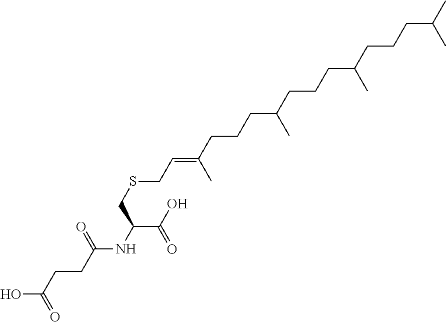 [SMILES: C/C(=C\CSC[C@H](NC(=O)CCC(=O)O)C(=O)O)CCCC(C)CCCC(C)CCCC(C)C]
or pharmaceutically acceptable salt or ester thereof, based on the total weight of (E)-4-((1-carboxy-2-((3,7,11,15-tetramethylhexadec-2-en-1-yl)thio)ethyl)amino)-4-oxobutanoic acid or a pharmaceutically acceptable salt or ester thereof present in the composition.

30. (canceled)

31. The pharmaceutical composition of claim 1, wherein the IPC Active Agent includes at least 97.5 wt % of 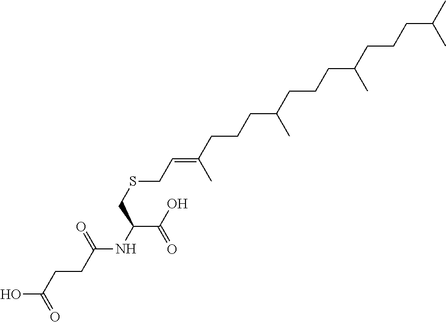 [SMILES: C/C(=C\CSC[C@H](NC(=O)CCC(=O)O)C(=O)O)CCCC(C)CCCC(C)CCCC(C)C]
or pharmaceutically acceptable salt or ester thereof, based on the total weight of (E)-4-((1-carboxy-2-((3,7,11,15-tetramethylhexadec-2-en-1-yl)thio)ethyl)amino)-4-oxobutanoic acid or a pharmaceutically acceptable salt or ester thereof present in the composition.

32. The pharmaceutical composition of claim 1, wherein the IPC Active Agent includes: 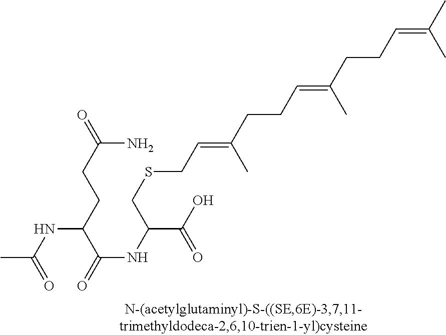 [SMILES: CC(=O)NC(CCC(N)=O)C(=O)NC(CSC/C=C(\C)CC/C=C(\C)CCC=C(C)C)C(=O)O]
or a pharmaceutically acceptable salt or ester thereof.

33. The pharmaceutical composition of claim 1, wherein the IPC Active Agent includes at least 75 wt % of: 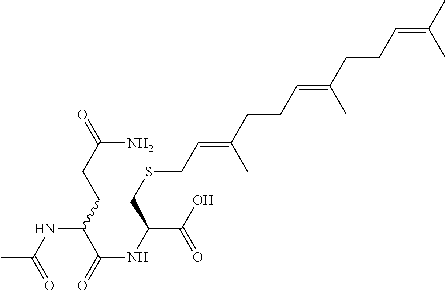 [SMILES: CC(=O)NC(CCC(N)=O)C(=O)N[C@@H](CSC/C=C(\C)CC/C=C(\C)CCC=C(C)C)C(=O)O]
or pharmaceutically acceptable salt or ester thereof, based on the total weight of N-(acetylglutaminyl)-S-((2E,6E)-3,7,11-trimethyldodeca-2,6,10-trien-1-yl)cysteine or a pharmaceutically acceptable salt or ester thereof present in the composition.

34-35. (canceled)

36. The pharmaceutical composition of claim 1, wherein the IPC Active Agent includes at least 90 wt % of: 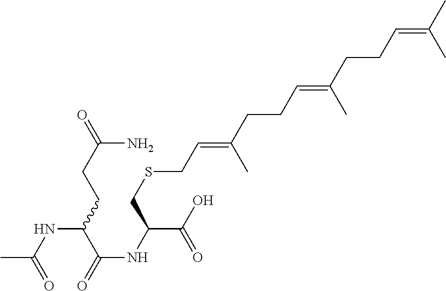 [SMILES: CC(=O)NC(CCC(N)=O)C(=O)N[C@@H](CSC/C=C(\C)CC/C=C(\C)CCC=C(C)C)C(=O)O]
or pharmaceutically acceptable salt or ester thereof, based on the total weight of N-(acetylglutaminyl)-S-((2E,6E)-3,7,11-trimethyldodeca-2,6,10-trien-1-yl)cysteine or a pharmaceutically acceptable salt or ester thereof present in the composition.

37. (canceled)

38. The pharmaceutical composition of claim 1, wherein the IPC Active Agent includes at least 97.5 wt % of: 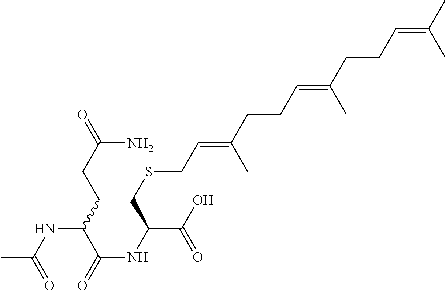 [SMILES: CC(=O)NC(CCC(N)=O)C(=O)N[C@@H](CSC/C=C(\C)CC/C=C(\C)CCC=C(C)C)C(=O)O]
or pharmaceutically acceptable salt or ester thereof, based on the total weight of N-(acetylglutaminyl)-S-((2E,6E)-3,7,11-trimethyldodeca-2,6,10-trien-1-yl)cysteine or a pharmaceutically acceptable salt or ester thereof present in the composition.
